(19) United States
(12) Patent Application Publication
(10) Pub. No.: US 2011/0229445 A1
(43) Pub. Date: Sep. 22, 2011

(54) METHOD FOR HEALING BONE FRACTURE USING TRANSFECTED CHONDROCYTES

(76) Inventors: Kwan Hee LEE, Rockville, MD (US); Moon Jong Noh, Rockville, MD (US); Youngsuk Yi, Rockville, MD (US)

(21) Appl. No.: 13/053,038

(22) Filed: Mar. 21, 2011

Related U.S. Application Data
(60) Provisional application No. 61/315,907, filed on Mar. 20, 2010.

(51) Int. Cl. A61K 35/32; A61P 19/08

(52) U.S. Cl. 424/93.21

(57) ABSTRACT

The application discloses a method for making bone at a bone defect site for a person suffering from low bone mass which includes inserting a gene encoding a protein having bone regenerating function into a connective tissue cell operably linked to a promoter, and transplanting the mammalian cell into the bone defect site, and allowing the bone defect site to make the bone.

### BACKGROUND OF THE INVENTION

1. Field of the Invention

The present invention relates to a method of introducing at least one gene encoding a member of the transforming growth factor β superfamily into at least one mammalian connective tissue cell for use in generating or regenerating bone, in particular, to repair fracture in osteoporotic bone or to fuse spine in the mammalian host.

2. Brief Description of the Related Art

Homeostasis of living bone tissue is a dynamic process modulated by regulatory signals such as hormones, and growth and differentiation factors. The growth factors known to stimulate proliferation of bone cells are bone morphogenic proteins (BMPs), transforming growth factor-β proteins (TGF-β), insulin-like growth factors (IGFs), and basic fibroblast growth factors (bFGFs).

Osteoporosis, which is characterized by low bone mass and microarchitectural deterioration of bone structure resulting in bone fractures, is a common health problem among increasing number of the elderly. Osteoporotic conditions also may be caused by a variety of factors, such as but not limited to menopause, calcium deficient diet, ovariectomization, glucocorticoid-induced osteoporosis, hyperthyroidism, immobilization, heparin-induction or immuno suppressive-induction.

Fracture healing is a complex process and remains poorly understood. In rat model produced by ovariectomy and low calcium diet to simulate patients with osteoporosis, fractured osteoporotic bone was not healed properly (Kubo et al., Steroid Biochemistry & Molecular Biology, 68:197-202, 1999; Namkung-Matthai et al., Bone, 28:80-86, 2001). Thus, therapies involving bone regeneration will also greatly improve the treatment of osteoporotic bone fracture.

In the orthopedic field, some cytokines have been considered to be candidates for the treatment of orthopedic diseases. Bone morphogenetic protein has been considered to be an effective stimulator of bone formation (Ozkaynak et al., EMBO J, 9:2085-2093, 1990; Sampath and Rueger, Complications in Ortho, 101-107, 1994), and TGF-β has been reported as a stimulator of osteogenesis and chondrogenesis (Joyce et al., J Cell Biology, 110:2195-2207, 1990). Transforming growth factor-β (TGF-β) is considered to be a multifunctional cytokine (Sporn and Roberts, Nature (London), 332: 217-219, 1988), and plays a regulatory role in cellular growth, differentiation and extracellular matrix protein synthesis (Madri et al., J Cell Biology, 106: 1375-1384, 1988). TGF-β inhibits the growth of epithelial cells and osteoclast-like cells in vitro (Chenu et al., Proc Natl Acad Sci, 85: 5683-5687, 1988), but it stimulates enchondral ossification and eventually bone formation in vivo (Critchlow et al., Bone, 521-527, 1995; Lind et al., A Orthop Scand, 64 (5): 553-556, 1993; and Matsumoto et al., In vivo, 8: 215-220, 1994). TGF-β-induced bone formation is mediated by its stimulation of the subperiosteal pluripotent cells, which eventually differentiate into cartilage-forming cells (Joyce et al., J Cell Biology, 110: 2195-2207, 1990; and Miettinen et al., J Cell Biology, 127-6: 2021-2036, 1994).

The biological effect of TGF-β in orthopedics has been reported (Andrew et al., Calcif Tissue In. 52: 74-78, 1993; Borque et al., Int J Dev Biol., 37:573-579, 1993; Carrington et al., J Cell Biology, 107:1969-1975, 1988; Lind et al., A Orthop Scand. 64 (5):553-556, 1993; Matsumoto et al., In vivo, 8:215-220, 1994). In mouse embryos, staining shows that TGF-β is closely associated with tissues derived from the mesenchyme, such as connective tissue, cartilage and bone. In addition to embryologic findings, TGF-β is present at the site of bone formation and cartilage formation. It can also enhance fracture healing in rabbit tibiae. Recently, the therapeutic value of TGF-β has been reported (Critchlow et al., Bone, 521-527, 1995; and Lind et al., A Orthop Scand, 64 (5): 553-556, 1993), but its short-term effects and high cost have limited wide clinical application.

Many bony deficits that are excessively traumatic will not result in complete recovery and will require therapeutic intervention(s) such as autografting or grafting from banked bone. A high rate of failure has been associated with these conventional therapies. And most of the recent alternative approaches utilize implantation of a biodegradable carrier impregnated with osteoinductive proteins to the injured site. See for example, U.S. Pat. No. 5,656,598. Other approaches include using a mechanical device to allow the bone to regenerate, such as disclosed in U.S. Pat. Nos. 6,077,076 and 6,022,349. One major disadvantage of these methods is the requirement of a large amount of recombinant proteins to achieve therapeutic effects due to the short duration of action of the therapeutic proteins in vivo.

An estimated 20 to 25 million people are at an increased risk of developing bone fractures due to the loss of bone mass that occurs in osteoporosis. Fractures in elderly individuals often require surgery and can lead to increased morbidity and mortality. The failure rate of conventional internal fixation is high in osteoporotic bone fractures, due to the decreased holding power of plate-and-screw fixation.¹ Although autogenous cancellous bone from the iliac crest provides the best healing power for bone fractures, surgeons were forced to use bone allografts because of complications.² The shortage of allograft donors spurred the development of three categories of synthetic graft substitutes.^{3,4} Osteoconductive matrix materials provide a microenvironment that supports growth of osteoprogenitor tissue, but they do not actively stimulate the bone formation process. Injectable osteoconductive calcium phosphate cements improved the holding strength of the metal devices.⁵ Osteoinductive bone graft substitutes actively participate in bone formation by triggering the recruitment of osteoprogenitor cells such as mesenchymal cells to the fracture site and differentiating the precursor cells into osteoblasts.⁶ BMPs promote differentiation of mesenchymal cells into chondrocytes and osteoblasts⁷ and differentiate osteoprogenitor cells such as muscle tissue into osteoblasts at the site of induced bone defects and ectopic locations.^{8,9}

Currently, BMP2 and BMP7 are approved to treat open tibial fractures and tibial non-unions. In clinical testing, recombinant human BMP7 and autologous bone grafts were comparable when analyzed using several clinical outcome parameters. However, the morbidity and pain associated with surgical harvesting of autologous bone were eliminated with the use of BMP.^{10, 11} The addition of recombinant human BMP2 to the treatment of type-III open fractures significantly reduced the frequency of bone-grafting procedures and other secondary interventions.¹¹ Further, recombinant human BMP2 produced higher rates of fusion and improved neurologic status and back and leg pain compared with the control group in single-level anterior lumbar interbody fusion.¹² BMP7 also improved the performance of autologous and allogeneic iliac graft and reduced the healing time.¹³

Bone marrow cells have been used to provide cells with osteogenic potential for use in osteogenic bone grafts.¹⁴ Autologous BMP2-producing bone marrow cells successfully healed a substantial femoral segmental defect in syngeneic rats.¹⁵ In that study, the investigators discovered that continuous expression of BMP2 was more effective for achieving regeneration than one application of BMP2 protein at the defect site. This is due, at least in part, to the short half-life of BMPs, and, therefore, efficacy is limited without repeated administration. To increase the potential efficacy of BMPs, a method of sustained delivery must be explored. Gene therapy methods are attractive alternatives for addressing these requirements.

A cell-mediated gene therapy to sustain TGF-β protein expression in vivo successfully regenerated cartilage¹⁶ when treating degenerative arthritis and similar diseases. We were interested in using the retrovirally transduced cells to provide BMP2 continuously for a limited time as an osteoinductive material from the cells transduced with the retroviral vector containing the BMP2 gene. Chondrocytes were selected as target cells for retroviral vector infection and single clones were selected after infection to obtain a cell that can secrete BMP2 at a constant rate of expression with continued subcultures. The BMP2-producing single clone was irradiated before injection to increase the safety of the retrovirally transduced cells by rendering them replication incompetent. Therefore, the amount of BMP2 secreted from the irradiated BMP2-producing cells was controllable within a certain range, and it was sufficient to precipitate bone regeneration in the fractures.

Bone deterioration in the vertebrae of the spine is another area where generating bone to fuse the vertebrae will provide relief to patients suffering from back pain caused by collapsed vertebrae. Therefore, there is a need in the art of therapeutic application for improving the length of release of osteogenic proteins. As described in this application, the present invention provides a method for the sustained expression of such an osteogenic therapeutic protein at the bone defect site leading to an efficient regeneration of bone.

### SUMMARY OF THE INVENTION

The present invention has met the hereinbefore described need. A method of introducing at least one gene encoding a product into at least one cell of a mammalian connective tissue for use in treating a mammalian host is provided in the present invention. This method includes employing recombinant techniques to produce a DNA vector molecule containing the gene coding for the product and introducing the DNA vector molecule containing the gene coding for the product into the connective tissue cell. The DNA vector molecule can be any DNA molecule capable of being delivered and maintained within the target cell or tissue such that the gene encoding the product of interest can be stably expressed. The DNA vector molecule preferably utilized in the present invention is either a viral or plasmid DNA vector molecule. This method preferably includes introducing the gene encoding the product into the cell of the mammalian connective tissue for therapeutic use.

The present invention is directed to a method for making bone at a bone defect site for a subject optionally suffering from low bone mass comprising:

a) inserting a gene encoding a protein having bone regenerating function into a vector operatively linked to a promoter, and

b) transfecting or transducing a population of connective tissue cells in vitro with said recombinant vector; and

c) transplanting the mammalian cell into the bone defect site, and allowing the bone defect site to make the bone.

In this method, the vector may be without limitation a retroviral vector or a plasmid vector. The gene may be a member of TGF-β superfamily, and in particular may be a bone morphogenetic protein (BMP). Further in particular, the BMP may be BMP-2. In addition, the connective tissue cell may be a fibroblast, chondrocyte or a bone progenitor cell or a combination thereof.

In the method above, the bone is generated during early period or late period after fracture.

The present invention is also directed to a method of fusing a spine, comprising:

a) inserting a gene encoding a protein having bone generating or regnerating function into a vector;

b) transfecting or transducing a population of connective tissue cells in vitro with said recombinant vector; and

c) contacting, transplanting or injecting an osteogenic effective amount of the transfected or transduced population of connective tissue cells and a pharmaceutically acceptable carrier thereof with the spine such that expression of the DNA sequence encoding the gene at the spine results in the generation of bone, whereby the spine is fused.

In this method, the vector may be without limitation a retroviral vector or a plasmid vector. The gene may be a member of TGF-β superfamily, and in particular may be a bone morphogenetic protein (BMP). Further in particular, the BMP may be BMP-2. In addition, the connective tissue cell may be a fibroblast, chondrocyte, a bone progenitor cell or a combination thereof.

In the method above, the bone is generated during early period or late period after fracture.

In addition, the invention is also directed to a method of healing osteoporotic fracture comprising:

a) inserting a gene encoding a protein having bone regenerating function into a vector,

b) transfecting or transducing a population of connective tissue cells in vitro with said recombinant vector; and

c) introducing the connective tissue cell into the fracture site, and allowing the fracture to heal.

In this method, the vector may be without limitation a retroviral vector or a plasmid vector. The gene may be a member of TGF-β superfamily, and in particular may be a bone morphogenetic protein (BMP). Further in particular, the BMP may be BMP-2. In addition, the connective tissue cell may be a fibroblast, chondrocyte, a bone progenitor cell or a combination thereof.

In the method above, the bone is generated during early period or late period after fracture. In all of the above-described methods, the cells employed may be preferably allogeneic relative to the host mammal.

These and other objects of the invention will be more fully understood from the following description of the invention, the referenced drawings attached hereto and the claims appended hereto.

### BRIEF DESCRIPTION OF THE DRAWINGS

The present invention will become more fully understood from the detailed description given herein below, and the accompanying drawings which are given by way of illustration only, and thus are not limitative of the present invention, and wherein;

FIGS. 1A and 1B show construction of pMT-BMP2 harboring human BMP2 gene.

FIGS. 2A-2F show regeneration of bone with NIH3T3-BMP-2 fibroblast cells. FIGS. 2A and 2B show pictures of leg bones after 8 weeks of injection of control NIH3T3 fibroblast cells (A) and NIH3T3-BMP-2 cells (B). FIGS. 2C-2F show radiographic examinations of the control (C & D) and experimental (E & F) leg bones before sacrificing the animals. The bone defect treated with cells expressing BMP-2 proteins healed after 8 weeks of injection.

FIGS. 3A-3D show histological examination of regenerated bone tissue. Paraffin sections of the regenerated bone tissue were made and stained with Mason's trichrome. The results showed that the structure of regenerated bone tissue (RB) was almost identical to that of the normal bone tissue (NB). FIGS. 3A and 3B show low magnifications (40×), and FIGS. 3C and 3D show high magnifications (100×). The dotted line indicates the borderline between the regenerated and normal bone tissue.

FIGS. 4A-4I show regeneration of bone with NIH3T3-hBMP2 fibroblast cells. NIH3T3-BMP-2 cells (2 ml of 2×10⁶ cells/ml) were injected into the defect area in the tibia bone after suturing. (A to G) Radiographic analysis was performed at 1, 2, 3, 4, 5, 6, and 7 weeks after injection of the cells. (H) The specimen was harvested at 7 weeks post injection and a picture was taken. (I) Histological examination was carried out after harvest. The result of Mason's trichrome staining is shown.

FIGS. 5A-5I show regeneration of bone with control DMEM medium. Control DMEM culture medium (2 ml) was injected into the defect area in the tibia bone after suturing. (A to G) Radiographic analysis was performed at 1 day, 1, 2, 3, 4, 5, and 6 weeks after injection of the medium. (H) The specimen was harvested at 6 weeks post injection and a picture was taken. (I) Histological examination was performed after harvest. The result of Mason's trichrome staining is shown.

FIGS. 6A and 6B show radiographs from rat TG001, 4 and 6 weeks after posterolateral intertransverse process fusion procedure implanting cells using absorbable collagen sponge (ACS) carrier. Radiographic bridging bone on left side is encircled after 5×10⁶ fibroblasts (mouse) transfected with cDNA for BMP-2 posterolateral intertransverse process fusion study. Note less cells probably on right side and less bone formation, if any.

FIGS. 7A-7C show use of cells transfected with BMP gene. (A) Schematic representation of the retroviral vector that contains human BMP2, BMP3, BMP4, BMP7, and BMP9 cDNA. Human BMPs cDNAs were cloned and inserted into the retroviral vector pMTMLV. Bp, base pair (B) Histology of ectopic bones formed by injection of cells producing BMP2 with or without normal chondrocytes in nude mice. (a) Fibrous tissue and hypertrophic cells has formed 6 weeks after injection of NIH3T3MT-BMP2 only. (b) Cartilage tissue has formed 6 weeks after injection of NIH3T3MT-BMP2 and normal chondrocytes. (c) Fibrous tissue is the major formation 8 weeks after injection of only NIH3T3MT-BMP2. (d) An osteoid has formed 8 weeks after injection of NIH3T3MT-BMP2 and normal chondrocytes. Six weeks after injection, sections were stained with safranin O (a, b). Eight weeks after injection, the sections were stained with hematoxylin and eosin (H&E) (c, d) (magnification, ×100). (C) RT-PCR analysis of HLA-a was performed. cDNA was obtained from chondrocyte cultures until passage 8. RT-PCR for glyceraldehyde 3-phosphate dehydrogenase was performed as the negative control.

FIGS. 8A-8B show characterization of selected single clones. (A) Growth characteristics of selected BMP2-expressing single clones (hChonJ-BMP2A and hChonJ-BMP2E) are tested with the increasing number of passages. The unit of cell numbers is 1×10⁵ (B) Expression of BMP2 protein in selected single clones (hChonJ-BMP2A and hChonJ-BMP2E) is measured with the increasing number of passages. The unit of cell numbers is pg/1×10⁵ cells/day.

FIGS. 9A-9C show measurement of BMP2 protein in selected irradiated single clones (hChonJ-BMP2A and hChonJ-BMP2E) to identify the lethal dosage. (A) Selected single clones were irradiated with either 10, 13, 15, 17, 20, or 25Gy of gamma rays. After the cells were incubated 4, 8, 12, 16, 20, 24, 28, and 32 days, the amount of BMP2 was determined with ELISA. BMP2 secretion increased until 2 weeks after injection with all radiation doses. The BMP2 expression level then decreased gradually until it reached undetectable levels about 1 month after irradiation. (B) Comparison of the total secreted BMP2 protein from the irradiated and non-irradiated BMP2-producing single clone (hChonJ-BMP2E). The total irradiated hChonJ-BMP2E is shown in pink below the graph of BMP2 secretion. The total non-irradiated hChonJ-BMP2E is seen in blue based on the assumption of no apparent growth after the injection.

FIGS. 10A-10B show generation of osteoporotic mice. (A) Micro-CT of the osteoporotic mice. Group 1 represents normal rats fed a regular diet, group 2 normal rats fed a diet containing AIN76A without calcium and phosphate, and group 3 ovariectomized rats fed with a diet containing AIN76A. Compared with the control rats (group 1), groups 2 and 3 started to show decreased bone mass from 5 weeks after the start of the AIN76A diet (compare white arrows at 5 weeks). (B) A comparison of BMD between the ovariectomized rats fed a low-calcium diet and the ovariectomized rats fed a calcium-free diet for 7 weeks. The BMD of the ovariectomized rats fed a calcium-free diet decreased continuously, while the BMD of the ovariectomized rats fed a low-calcium diet increased continuously. W, weeks.

FIGS. 11a-11t show radiologic evidence of healing of the fractures is seen in the osteoporotic mice after the injection of irradiated and non-irradiated BMP2-producing cells. For the injection of cells, 2×10⁶ cells were used. For the injection of BMP2 protein, 80 μg of protein was used. BMP2-producing human chondrocytes were injected. Samples were injected into the fracture sites of the osteoporotic rats, and X-rays were taken 0, 2, 4, and 6 weeks after injection. The metal pins that were inserted appear black in the picture.

FIGS. 12a-12t show histologic examination of bones at the fracture sites. Samples were harvested at 4, 6, 8, and 12 weeks after the injection and stained using Masson's trichrome for microscopic analysis of the regenerated bone. Four weeks after injection, callus has formed and the fracture gap is bridged by the cartilage mass and some woven bone (a, b, c, d, e). After irradiated hChonJ-BMP2E was injected, most of the gap was replaced with bone by 4 weeks later (d); areas of cartilage are still present in the filled gaps (a, b, c, e). Six weeks after injection, bridging of the gap with bone is almost finished at the site of the injection of irradiated hChonJ-BMP2E and there is continuous bone marrow across the fracture site (i). The final shapes of the bones in the rats injected with irradiated hChonJ-BMP2E are similar morphologically to the original bones before the fractures were created (n, s) (magnification, ×50). The red arrows indicate the areas of interest. W, weeks.

FIGS. 13a-13o show gross bones near the fracture sites. Samples were harvested at 4, 6, 8, and 12 weeks after the injection and images near the fracture sites were obtained to examine the outer part of the bone structure. The shapes of the bones in the rats injected with irradiated hChonJ-BMP2E were morphologically similar to the original bones before the fractures were created (d, i, n). When only BMP2 protein was injected (80 μg), extra tissue, which are soft with numerous pores, formed around the callus (e, j, o). The red arrows indicate the areas of interest. W, weeks.

FIGS. 14A-14D show comparison of doses. Before injection, 2×10⁵, 5×10⁶, and 2×10⁶ cells of clone hChonJ-BMP2E were irradiated with 10 Gy. Radiogram and computer-aided three-dimensional bone images were analyzed biweekly until week 12. (A) PBS was injected. An average time to the regeneration of fractured bone was 11.5 weeks. (B) Irradiated hChonJ-BMP2E (2×10⁵) was injected. It took about 9 weeks for complete repair of the fractured bone. (C) Irradiated hChonJ-BMP2E (5×10⁶) was injected. An average of 8.8 weeks was required for fracture repair. (D) Injection of irradiated hChonJ-BMP2E (2×10⁶) resulted in the shortest time (7.6 weeks) to full recovery of the fractures. W, weeks.

### DETAILED DESCRIPTION OF THE INVENTION

In the present application, “a” and “an” are used to refer to both single and a plurality of objects.

As used herein, administration “in combination with” one or more further therapeutic agents includes simultaneous (concurrent) and consecutive administration in any order.

As used herein, the term “biologically active” in reference to a nucleic acid, protein, protein fragment or derivative thereof is defined as an ability of the nucleic acid or amino acid sequence to mimic a known biological function elicited by the wild type form of the nucleic acid or protein.

As used herein, the term “bone growth” relates to bone mass. TGF-β protein is thought to increase bone mass systemically. This is suggested by the increase in the number and size of osteoblasts, and increased deposition of osteoid lining bone surfaces following systemic administration.

As used herein, “carriers” include pharmaceutically acceptable carriers, excipients, or stabilizers which are nontoxic to the cell or mammal being exposed thereto at the dosages and concentrations employed. Often, the pharmaceutically acceptable carrier is an aqueous pH buffered solution. Examples of pharmaceutically acceptable carriers include without limitation buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONICS®.

As used herein, the term “connective tissue” is any tissue that connects and supports other tissues or organs, and includes but is not limited to a ligament, a cartilage, a tendon, a bone, or a synovium of a mammalian host.

As used herein, the term “connective tissue cell” or “cell of a connective tissue” include cells that are found in the connective tissue, such as fibroblasts, cartilage cells (chondrocytes), and bone cells (osteoblasts/osteocytes), as well as fat cells (adipocytes) and smooth muscle cells. Preferably, the connective tissue cells are fibroblasts, chondrocytes, and bone cells. More preferably, the connective tissue cells are fibroblast cells. Alternatively, the connective tissue cells are osteoblast or osteocytes. It will be recognized that the invention can be practiced with a mixed culture of connective tissue cells, as well as cells of a single type. It is also recognized that the tissue cells may be treated such as by chemical or radiation so that the cells stably express the gene of interest. Preferably, the connective tissue cell does not cause a negative immune response when injected into the host organism. It is understood that allogeneic cells may be used in this regard, as well as autologous cells for cell-mediated gene therapy or somatic cell therapy.

As used herein, “connective tissue cell line” includes a plurality of connective tissue cells originating from a common parent cell.

As used herein, “host cell” includes an individual cell or cell culture which can be or has been a recipient of a vector of this invention. Host cells include progeny of a single host cell, and the progeny may not necessarily be completely identical (in morphology or in total DNA complement) to the original parent cell due to natural, accidental, or deliberate mutation and/or change. A host cell includes cells transfected or infected in vivo with a vector comprising a polynucleotide encoding a member of the TGF-β superfamily.

As used herein, the term, “low bone mass” refers to a condition where the level of bone mass is below the age specific normal as defined in standards by the World Health Organization “Assessment of Fracture Risk and its Application to Screening for Postmenopausal Osteoporosis (1994). Report of a World Health Organization Study Group. World Health Organization Technical Series 843”, which is incorporated by reference herein in its reference to normal and osteoporotic levels of bone mass. Further, the term “bone mass” refers to bone mass per unit area, which is sometimes referred to as bone mineral density.

As used herein, the term “maintenance”, when used in the context of liposome delivery, denotes the ability of the introduced DNA to remain present in the cell. When used in other contexts, it means the ability of targeted DNA to remain present in the targeted cell or tissue so as to impart a therapeutic effect.

As used herein, the term “mammalian host” includes members of the animal kingdom including but not limited to human beings.

As used herein, the term “mature bone” relates to bone that is mineralized, in contrast to non-mineralized bone such as osteoid.

As used herein, the term “osteogenically effective” means that amount which effects the formation and development of mature bone.

As used herein, the term “osteoprogenitor cells” or “bone progenitor cells” are cells that have the potential to become bone cells, and reside in the periosteum and the marrow. Osteoprogenitor cells are derived from connective tissue progenitor cells that reside also in the surrounding tissue (muscle).

As used herein, the term “patient” includes members of the animal kingdom including but not limited to human beings.

As used herein, a composition is “pharmacologically or physiologically acceptable” if its administration can be tolerated by a recipient animal and is otherwise suitable for administration to that animal. Such an agent is said to be administered in a “therapeutically effective amount” if the amount administered is physiologically significant. An agent is physiologically significant if its presence results in a detectable change in the physiology of a recipient patient.

As used herein “pharmaceutically acceptable carrier and/or diluent” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

As used herein, a “promoter” can be any sequence of DNA that is active, and controls transcription in an eucaryotic cell. The promoter may be active in either or both eucaryotic and procaryotic cells. Preferably, the promoter is active in mammalian cells. The promoter may be constitutively expressed or inducible. Preferably, the promoter is inducible. Preferably, the promoter is inducible by an external stimulus. More preferably, the promoter is inducible by hormones or metals. Likewise, “enhancer elements”, which also control transcription, can be inserted into the DNA vector construct, and used with the construct of the present invention to enhance the expression of the gene of interest.

As used herein, “selectable marker” includes a gene product that is expressed by a cell that stably maintains the introduced DNA, and causes the cell to express an altered phenotype such as morphological transformation, or an enzymatic activity. Isolation of cells that express a transfected gene is achieved by introduction into the same cells a second gene that encodes a selectable marker, such as one having an enzymatic activity that confers resistance to an antibiotic or other drug. Examples of selectable markers include, but are not limited to, thymidine kinase, dihydrofolate reductase, aminoglycoside phosphotransferase, which confers resistance to aminoglycoside antibiotics such as kanamycin, neomycin and geneticin, hygromycin B phosphotransferase, xanthine-guanine phosphoribosyl transferase, CAD (a single protein that possesses the first three enzymatic activities of de novo uridine biosynthesis—carbamyl phosphate synthetase, aspartate transcarbamylase and dihydroorotase), adenosine deaminase, and asparagine synthetase (Sambrook et al. Molecular Cloning, Chapter 16. 1989), incorporated herein by reference in its entirety.

As used herein, “subject” is a vertebrate, preferably a mammal, more preferably a human.

As used herein, “treatment” is an approach for obtaining beneficial or desired clinical results. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. “Treatment” refers to both therapeutic treatment and prophylactic or preventative measures. Those in need of treatment include those already with the disorder as well as those in which the disorder is to be prevented. “Palliating” a disease means that the extent and/or undesirable clinical manifestations of a disease state are lessened and/or the time course of the progression is slowed or lengthened, as compared to a situation without treatment.

As used herein, “TGF-β protein” refers to a member of the TGF-β superfamily of proteins.

As used herein, “vector”, “polynucleotide vector”, “construct” and “polynucleotide construct” are used interchangeably herein. A polynucleotide vector of this invention may be in any of several forms, including, but not limited to, RNA, DNA, RNA encapsulated in a retroviral coat, DNA encapsulated in an adenovirus coat, DNA packaged in another viral or viral-like form (such as herpes simplex, and adeno-associated virus (AAV)), DNA encapsulated in liposomes, DNA complexed with polylysine, complexed with synthetic polycationic molecules, complexed with compounds such as polyethylene glycol (PEG) to immunologically “mask” the molecule and/or increase half-life, or conjugated to a non-viral protein. Preferably, the polynucleotide is DNA. As used herein, “DNA” includes not only bases A, T, C, and G, but also includes any of their analogs or modified forms of these bases, such as methylated nucleotides, internucleotide modifications such as uncharged linkages and thioates, use of sugar analogs, and modified and/or alternative backbone structures, such as polyamides.

Transforming Growth Factor-β (TGF-β) Superfamily

Transforming growth factor-β (TGF-β) superfamily encompasses a group of structurally related proteins, which affect a wide range of differentiation processes during embryonic development. This is based on primary amino acid sequence homologies including absolute conservation of seven cysteine residues. The family includes, Müllerian inhibiting substance (MIS), which is required for normal male sex development (Behringer, et al., Nature, 345:167, 1990), Drosophila decapentaplegic (DPP) gene product, which is required for dorsal-ventral axis formation and morphogenesis of the imaginal disks (Padgett, et al., Nature, 325:81-84, 1987), the Xenopus Vg-1 gene product, which localizes to the vegetal pole of eggs (Weeks, et al., Cell, 51:861-867, 1987), the activins (Mason, et al., Biochem, Biophys. Res. Commun, 135:957-964, 1986), which can induce the formation of mesoderm and anterior structures in Xenopus embryos (Thomsen, et al., Cell, 63:485, 1990), and the bone morphogenetic proteins (BMP's, such as BMP-2 to BMP-15) which can induce de novo cartilage and bone formation (Sampath, et al., J. Biol. Chem., 265:13198, 1990). The TGF-β gene products can influence a variety of differentiation processes, including adipogenesis, myogenesis, chondrogenesis, hematopoiesis, and epithelial cell differentiation (for a review, see Massague, Cell 49:437, 1987), which is incorporated herein by reference in its entirety.

The proteins of the TGF-β family are initially synthesized as a large precursor protein, which subsequently undergoes proteolytic cleavage at a cluster of basic residues approximately 110-140 amino acids from the C-terminus. The C-terminal regions of the proteins are all structurally related and the different family members can be classified into distinct subgroups based on the extent of their homology. Although the homologies within particular subgroups range from 70% to 90% amino acid sequence identity, the homologies between subgroups are significantly lower, generally ranging from only 20% to 50%. In each case, the active species appears to be a disulfide-linked dimer of C-terminal fragments. For most of the family members that have been studied, the homodimeric species has been found to be biologically active, but for other family members, like the inhibins (Ung, et al., Nature, 321:779, 1986) and the TGF-β's (Cheifetz, et al., Cell, 48:409, 1987), heterodimers have also been detected, and these appear to have different biological properties than the respective homodimers.

Members of the superfamily of TGF-β genes include TGF-β3, TGF-β2, TGF-β4 (chicken), TGF-β1, TGF-β5 (Xenopus), BMP-2, BMP-4, Drosophila DPP, BMP-5, BMP-6, Vgr1, OP-1/BMP-7, Drosophila 60A, GDF-1, Xenopus Vgf, BMP-3, Inhibin-βA, Inhibin-βB, Inhibin-α, and MIS. These genes are discussed in Massague, Ann Rev. Biochem. 67:753-791, 1998, which is incorporated herein by reference in its entirety.

Preferably, the member of the superfamily of TGF-β genes is BMP. More preferably, the member is TGF-β1, TGF-β2, TGF-β3, BMP-2, BMP-3, BMP-4, BMP-5, BMP-6, or BMP-7. Even more preferably, the member is human BMP. Most preferably, the member is human BMP-2.

BMP

BMPs are proteins which act to induce the differentiation of mesenchymal-type cells into chondrocytes and osteoblasts before initiating bone formation. They promote the differentiation of cartilage- and bone-forming cells near sites of fractures but also at ectopic locations. Some of the proteins induce the synthesis of alkaline phosphatase and collagen in osteoblasts. Some BMPs act directly on osteoblasts and promote their maturation while at the same time suppressing myogenous differentiation. Other BMPs promote the conversion of typical fibroblasts into chondrocytes and are capable also of inducing the expression of an osteoblast phenotype in non-osteogenic cell types. The BMP family belonging to the TGF-β superfamily comprises:

BMP-2A or BMP-2-α (114 amino acids) has been renamed BMP-2. Human, mouse and rat proteins are identical in their amino acid sequences. The protein shows 68 percent homology with Drosophila.

BMP-2B or BMP-2-β (116 amino acids) has been renamed BMP-4. Mouse and rat proteins are identical in their protein sequences.

BMP-3 (110 amino acids) is a glycoprotein and is identical to Osteogenin Human and rat mature proteins are 98 percent identical.

BMP-3b (110 amino acids) is related to BMP-3 (82 percent identity). Human and mouse proteins show 97 percent identity (3 different amino acids). Human and rat protein sequences differ by two amino acids. The factor is identical with GDF-10.

BMP-4 is identical with BMP-2B and with DVR-4. The protein shows 72 percent homology with Drosophila.

BMP-5 (138 amino acids). At the amino acid level human and mouse proteins are 96 percent identical.

BMP-6 (139 amino acids) is identical with DVR-6 and vegetal-specific-related-1.

BMP-7 (139 amino acids) is identical with OP-1 (osteogenic protein-1). Mouse and human proteins are 98 percent identical. The mature forms of BMP-5, BMP-6, and BMP-7 show 75 percent identity.

BMP-8 (139 amino acids) is identical with OP-2. The factor is referred to also as BMP-8a.

BMP-8b (139 amino acids) is identical with OP-3 and has been found in mice only. The factor is known also as OP-3.

BMP-9 (110 amino acids) is also referred to as GDF-5.

BMP-10 (108 amino acids) has been isolated from bovine sources. Bovine and human proteins are identical.

BMP-11 (109 amino acids) has been isolated from bovine sources. Human and bovine sequences are identical. The protein is referred to also as GDF-11.

BMP-12 (104 amino acids) is known also as GDF-7 or CDMP-3.

BMP-13 (120 amino acids) is the same as GDF-6 and CDMP-2.

BMP-14 (120 amino acids) is the same as GDF-5 and CDMP-1.

BMP-15 (125 amino acids) is expressed specifically in the oocyte. The murine protein is most closely related to murine GDF-9.

Some of these proteins exist as heterodimers. OP-1, for example, associates with BMP-2A.

Because of the high degree of amino acid sequence homology (approximately 90 percent), BMP-5, BMP-6, and BMP-7 are recognized as a distinct subfamily of the BMPs. The genes encoding BMP-5 and BMP-6 map to human chromosome 6. The gene encoding BMP-7 maps to human chromosome 20. BMPs can be isolated from demineralized bones and osteosarcoma cells. They have been shown also to be expressed in a variety of epithelial and mesenchymal tissues in the embryo. Some BMPs (for example, BMP-2 and BMP-4) have been shown to elicit qualitatively identical effects (cartilage and bone formation) and to have the ability to substitute for one another.

Osteogenin and related BMPs also promote additional successive steps in the endochondral bone formation cascade by functioning as potent chemoattractants for circulating monocytes and by inducing, among other things, the synthesis and secretion of TGF-β1 by monocytes. Monocytes stimulated by TGF-β secrete a number of chemotactic and mitogenic cytokines into the conditioned medium that recruit endothelial and mesenchymal cells and promote the synthesis of collagen and associated matrix constituents.

Despite the well-known activity of BMPs in bone formation, there have been numerous contradictory reports about the efficacy of BMP2, BMP4, BMP7, and BMP9 on bone regeneration under many different experimental conditions. Treatment of degenerative spondylolisthesis via BMP7 putty had reasonable success.¹⁸ BMP6 and BMP9 exhibited the highest osteogenic activity in vitro and in vivo.¹⁹ BMP4 was found most in various parts of human bones.²⁰ BMP4 and BMP6 introduced into athymic rats using adenoviral vector showed the best bone formation.²¹ BMP2, BMP6, and BMP9 induced osteoblast differentiation of mesenchymal stem cells.²² To find the best BMP for purpose of osteoporotic bone regeneration in the current study, we tested various BMPs under the same conditions in mice. Chondrocytes were provided in addition to BMPs-producing cells to facilitate cartilage formation that converts to bone during endochondral ossification. The only group in which bone formation occurred was that in which BMP2-producing NIH3T3 with chondrocytes was injected. This was surprising because BMP7, which has been reported to be one of the bone inducers, was not associated with bone formation in our experiments.^{9,23}

BMP2-producing cells were used in the osteoporotic bone fracture healing experiment based on the promising data from BMP2-producing NIH3T3 in the nude mice experiment. In the osteoporotic bone fracture healing experiment, chondrocytes were transduced instead of NIH3T3. The BMP2-producing chondrocytes were injected into the fracture sites in rats without the untransduced chondrocytes used in the nude mice study, because it was expected that pre-osteoblastic cells could be recruited easily into the environment of tibial bone fracture.

Based on previous experience with allogeneic human chondrocytes that express transforming growth factor beta 1 (TGFβ1) that were generated with a retroviral vector,²⁴ the cell irradiation was chosen as a means to render the cells replication incompetent, as this method allows for continuing limited expression of the desired transgene while ensuring that the cells do not persist. This method is used in several cell and gene therapy products approved for use in clinical trials by the FDA including the aforementioned TGFβ1 expressing chondrocytes and various cancer therapies.²⁵

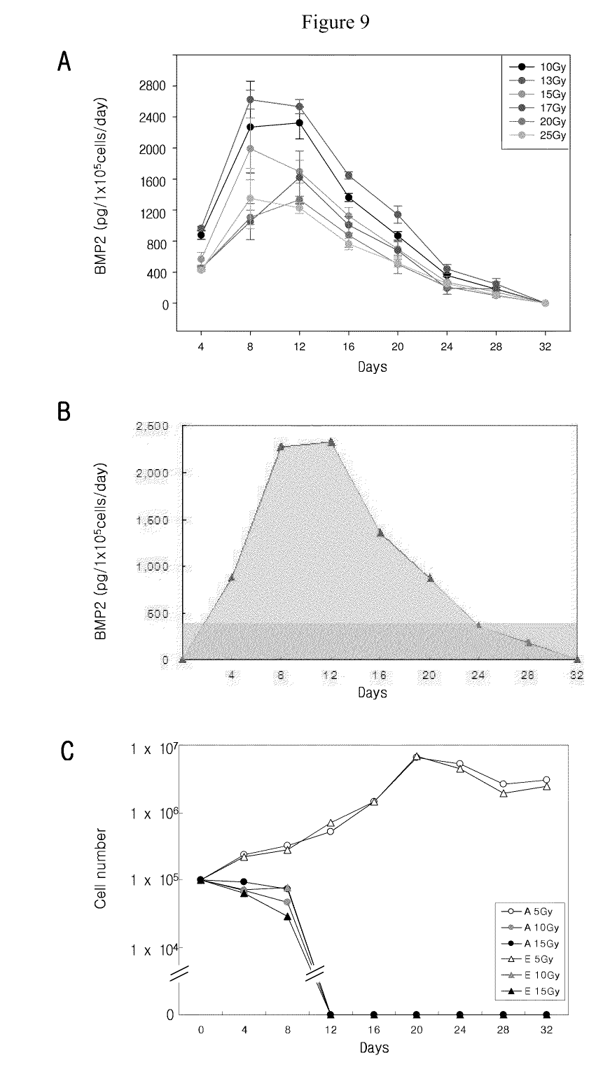

It was noteworthy that irradiated BMP2-producing chondrocytes showed a faster healing time compared with the non-irradiated ones. In addition, the irradiated BMP2-producing cells produced the typical callus shape. Three rats per group were used in the first experiment and five rats per group in the second experiment. The differences were consistent. The high amount of BMP2 secreted from irradiated BMP2-producing cells could be a reason for this unexpected result. The area below the BMP2 expression graph of the irradiated cells can be interpreted as the total amount of secreted BMP2 (shown in pink) (FIG. 9B). The calculated amount of BMP2 expression from the irradiated cells was 1.3 μg, which was much higher than 279 ng, secreted from the non-irradiated cells shown in blue. Even though there is a possibility of underestimation of BMP2 production from the non-irradiated cells, we suppose that the amount of BMP2 production should be low because most injected cells were not replicating well due to the hypertrophia and eventual calcification of the injected chondrocytes. An irradiated xenogeneic cell line engineered to secrete human interleukin-2 (IL-2) recently was reported to extend survival in mice with brain tumors.²⁵ In addition, articular cartilage was regenerated successfully with irradiated TGF-β-producing cells.²⁴ Considering our results and other similar reports described here, cell irradiation could be a way of providing necessary proteins when safe approaches are strictly required such as with gene therapy.

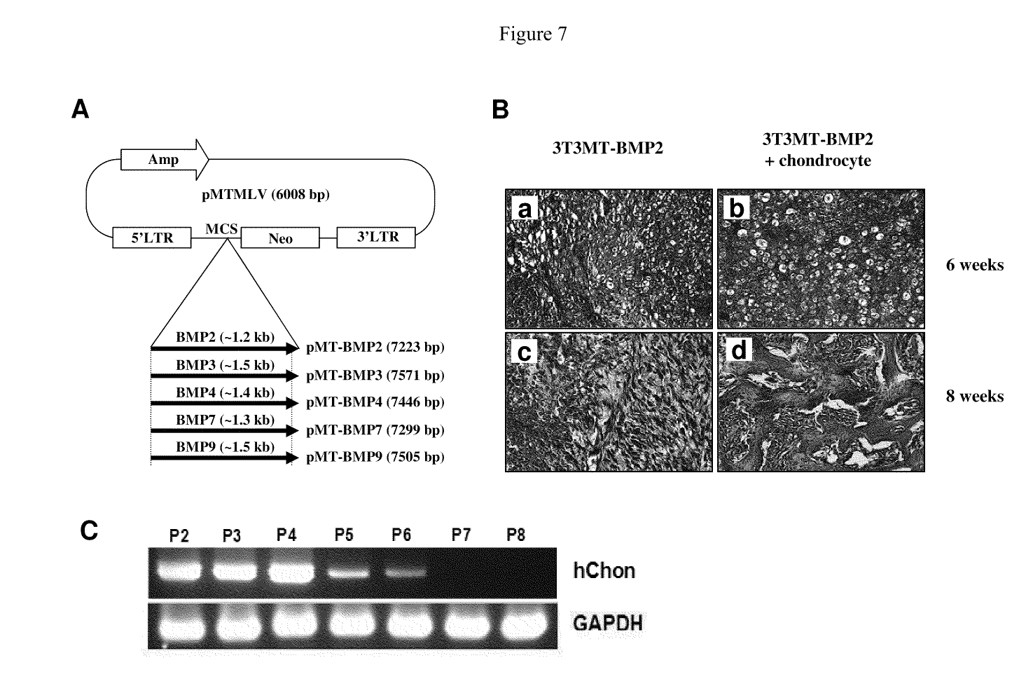

Xenogeneic chondrocytes were used for bone regeneration in this experiment. To show activity in this experiment, the BMP2-expressing xenogeneic chondrocytes would need to have escaped from immune response. Our observation of decreasing expression of HLA-a during the subculturing of chondrocytes in vitro could provide a clue to the avoiding the immune response (FIG. 7C). A report that showed that cytotoxic T lymphocytes could not gain access to chondrocytes due to the low level of major histocompatibility complex class I also support our results.²⁶

BMP2 recruits pluripotent cells from circulating blood to a vascularized coralline scaffold.²⁷ This recruitment of mesenchymal stem cells should have played a role in the fast progression of endochondral ossification in rats injected with BMP2-producing chondrocytes in our experiments. One plausible explanation for the good results obtained when normal human chondrocytes were injected (average, 6.7 weeks) is that the chondrocytes may have been involved in the regeneration of hyaline cartilage, which is the first step in endochondral bone formation.

Considering that the BMP2 expression level from the irradiated BMP2-producing clone (1.3 μg for hChonJ-BMP2E) is much less than the amount of BMP2 protein injected alone (80 μg), we surmise that the physical concentration of BMP2 needed for bone repair is much lower than that of the BMP2 protein we used. The opinion that BMPs production in situ, normal post-translational processing and presentation to the surrounding tissues occur in a natural cell manner is important and matches our results.²⁸ The bulky tissues that formed near the fracture site when BMP2 protein was injected were a side effect of overgrowth due to the higher BMP2 concentration. The tissues can even cause limited motion and eventually lead to myositis ossificans. Therefore, providing the BMP2 from the cell is a safer and more efficacious method.

In conclusion, we showed that irradiated BMP2-producing chondrocytes promote healing of osteoporotic bone fractures and the delivery of irradiated BMP2-producing chondrocytes could be an alternative method of direct BMP2 protein administration.

Therapy for Bone Regeneration

The present invention discloses ex vivo and in vivo techniques for delivery of a DNA sequence of interest to the connective tissue cells of the mammalian host. The ex vivo technique involves culture of target connective tissue cells, in vitro transfection of the DNA sequence, DNA vector or other delivery vehicle of interest into the connective tissue cells, followed by transplantation of the modified connective tissue cells to the target bone defect area of the mammalian host so as to effect in vivo expression of the gene product of interest.

It is to be understood that it is possible that substances such as scaffolding framework, matrix or bioadhesive such as buffy coat or other chemical adhesive, as well as various extraneous tissues and biocompatible carriers and other auxiliary materials may be implanted together with the genetically modified cells of the present invention. In one aspect, the invention may include bioadhesives in the therapeutic composition to facilitate contact between the genetically modified connective tissue cell and the area at or near the bone defect. Alternatively, it is possible that such substances may be excluded from the composition in the administration system of the invention.

It will be understood by the artisan of ordinary skill that the preferred source of cells for treating a human patient is the patient's own connective tissue cells, such as autologous fibroblast or osteoprogenitor cells (bone progenitor cells), osteocytes, osteoblasts or osteoclasts, but that allogeneic cells may also be used.

More specifically, this method includes employing a gene product that is a member of the transforming growth factor β superfamily, or a biologically active derivative or fragment thereof.

In another embodiment of this invention, a compound for parenteral administration to a patient in a therapeutically effective amount is provided that contains a TGF-β superfamily protein and a suitable pharmaceutical carrier.

Another embodiment of this invention provides for a compound for parenteral administration to a patient in a prophylactically effective amount that includes a TGF-β superfamily protein and a suitable pharmaceutical carrier.

In the present application, a method is provided for generating or regenerating bone by injecting an appropriate mammalian cell that is transfected or transduced with a gene encoding a member of the transforming growth factor-beta (TGF-β) superfamily, including, but not limited to, BMP-2 and TGF-β1, 2, and 3. BMP-2 is exemplified.

In an embodiment of the invention, it is understood that the cells may be injected into the area in which bone is to be generated or regenerated with or without scaffolding material or any other auxiliary material, such as extraneous cells or other biocompatible carriers. That is, the modified cells may be injected into the area in which bone is to be regenerated without the aid of any additional structure or framework. In one embodiment of the invention, such additional substances are disclosed in, for example, U.S. Pat. No. 5,842,477 and may be excluded from the composition of the invention.

The method of the present invention may be applied to all types of bones in the body, including but not limited to, non-union fractures (fractures that fail to heal), craniofacial reconstruction, segmental defect due to tumor removal, augmentation of bone around a hip implant revision (i.e., 25% of hip implants are replacements of an existing implant, as the lifespan of a hip implant is only ˜10 years), reconstruction of bone in the jaw for dental purposes. Further, other target bones include vertebrae on the spine for spine fusion, large bones, and so on.

The cells to be modified include any appropriate mammalian connective tissue cell, which assists in the formation of bone, including, but not limited to, fibroblast cells, osteoprogenitor cells, osteoblasts, osteocytes and osteoclasts, and may further include chondrocytes. However, it is understood that other non-genetically modified cells may also be included in the composition that is used to contact the bone defect site, such as osteoblasts, osteocytes, osteoclasts, chondrocytes, and so on.

As an alternative to the in vitro manipulation of the host cells, the gene encoding the product of interest is introduced into liposomes and injected directly into the area at or near the bone fracture or defect, where the liposomes fuse with the connective tissue cells, resulting in an in vivo gene expression of the gene product belonging to the TGF-β superfamily.

Where mention is made of “bone defect” or “defected bone”, it is to be understood that such defects may include fractures, breaks, and/or degradation of the bone including such conditions caused by injuries or diseases, and further may include defects in the spine vertebrae and further degradation of the disc area between the vertebrae. In one aspect of the invention, pain caused by the degradation of disk space between vertebrae may be treated by fusing vertebrae that surround the disk space that has degenerated.

As an additional alternative to the in vitro manipulation of connective tissue cells, the gene encoding the product of interest is introduced into the defected bone area as naked DNA. The naked DNA enters the connective tissue cell, resulting in an in vivo gene expression of the gene product belonging to the TGF-β superfamily.

One ex vivo method of treating a fractured or defected bone disclosed throughout this specification comprises initially generating a recombinant viral or plasmid vector which contains a DNA sequence encoding a protein or biologically active fragment thereof. This recombinant vector is then used to infect or transfect a population of in vitro cultured connective tissue cells, resulting in a population of connective tissue cells containing the vector. These connective tissue cells are then transplanted to a target bone defected area of a mammalian host, effecting subsequent expression of the protein or protein fragment within the defected area. Expression of this DNA sequence of interest is useful in substantially repairing the fracture or defect.

More specifically, this method includes employing as the gene a gene capable of encoding a member of the transforming growth factor β superfamily, or a biologically active derivative or fragment thereof and a selectable marker, or a biologically active derivative or fragment thereof.

A further embodiment of the present invention includes employing as the gene a gene capable of encoding at least one member of transforming growth factor β superfamily or a biologically active derivative or fragment thereof, and employing as the DNA plasmid vector any DNA plasmid vector known to one of ordinary skill in the art capable of stable maintenance within the targeted cell or tissue upon delivery, regardless of the method of delivery utilized.

Another embodiment of this invention provides a method for introducing at least one gene encoding a product into at least one cell of a connective tissue for use in treating the mammalian host. This method includes employing non-viral means for introducing the gene coding for the product into the connective tissue cell. More specifically, this method includes liposome encapsulation, calcium phosphate coprecipitation, electroporation, or DEAE-dextran mediation, and includes employing as the gene a gene capable of encoding a member of transforming growth factor superfamily or biologically active derivative or fragment thereof, and a selectable marker, or biologically active derivative or fragment thereof.

Another embodiment of this invention provides an additional method for introducing at least one gene encoding a product into at least one cell of a connective tissue for use in treating the mammalian host. This additional method includes employing the biologic means of utilizing a virus to deliver the DNA vector molecule to the target cell or tissue. Preferably, the virus is a pseudo-virus, the genome having been altered such that the pseudovirus is capable only of delivery and stable maintenance within the target cell, but not retaining an ability to replicate within the target cell or tissue. The altered viral genome is further manipulated by recombinant DNA techniques such that the viral genome acts as a DNA vector molecule which contains the heterologous gene of interest to be expressed within the target cell or tissue.

A preferred embodiment of the invention is a method of delivering TGF-β protein to a target defect area by delivering the TGF-β gene to the connective tissue of a mammalian host through use of a retroviral vector with the ex vivo technique disclosed within this specification. In other words, a DNA sequence of interest encoding a functional TGF-β protein or protein fragment is subcloned into a retroviral vector of choice, the recombinant viral vector is then grown to adequate titer and used to infect in vitro cultured connective tissue cells, and the transduced connective tissue cells, preferably autografted cells, are transplanted into the bone defect region or a therapeutically effective nearby area.

Another preferred method of the present invention involves direct in vivo delivery of a TGF-β superfamily gene to the connective tissue of a mammalian host through use of either an adenovirus vector, adeno-associated virus (AAV) vector or herpes-simplex virus (HSV) vector. In other words, a DNA sequence of interest encoding a functional TGF-β protein or protein fragment is subcloned into the respective viral vector. The TGF-β containing viral vector is then grown to adequate titer and directed into bone defect region or an osteogenically effective nearby area.

Methods of presenting the DNA molecule to the target connective tissue of the joint includes, but is not limited to, encapsulation of the DNA molecule into cationic liposomes, subcloning the DNA sequence of interest in a retroviral or plasmid vector, or the direct injection of the DNA molecule itself into the bone defect area or an osteogenically effective nearby area. The DNA molecule is preferably presented as a DNA vector molecule, either as recombinant viral DNA vector molecule or a recombinant DNA plasmid vector molecule. Expression of the heterologous gene of interest is ensured by inserting a promoter fragment active in eukaryotic cells directly upstream of the coding region of the heterologous gene. One of ordinary skill in the art may utilize known strategies and techniques of vector construction to ensure appropriate levels of expression subsequent to entry of the DNA molecule into the connective tissue.

It will be appreciated by those skilled in the art, that the viral vectors employing a liposome are not limited by cell division as is required for the retroviruses to effect infection and integration of connective tissue cells. This method employing non-viral means as hereinbefore described includes employing as the gene a gene capable of encoding a member belonging to the TGF-β superfamily and a selectable marker gene, such as an antibiotic resistance gene.

A further embodiment of this invention includes storing the connective tissue cell prior to transferring the cells. It will be appreciated by those skilled in the art that the connective tissue cell may be stored frozen in 10 percent DMSO in liquid nitrogen.

The inventors made stable fibroblast and chondrocyte cell lines by transfecting BMP-2 expression constructs. These BMP-2-producing cells maintained high concentration of active BMP-2 concentration in vivo for a long duration.

Therapy for Healing Osteoporotic Bone Fracture

Osteoporosis is a structural deterioration of the skeleton caused by loss of bone mass resulting from an imbalance in bone formation, bone resorption, or both, such that resorption dominates the bone formation phase, thereby reducing the weight-bearing capacity of the affected bone. In a healthy adult, the rate at which bone is formed and resorbed is tightly coordinated so as to maintain the renewal of skeletal bone. However, in osteoporotic individuals an imbalance in these bone remodeling cycles develops which results in both loss of bone mass and in formation of microarchitectural defects in the continuity of the skeleton. These skeletal defects, created by perturbation in the remodeling sequence, accumulate and finally reach a point at which the structural integrity of the skeleton is severely compromised and bone fracture is likely. Although this imbalance occurs gradually in most individuals as they age (“senile osteoporosis”), it is much more severe and occurs at a rapid rate in postmenopausal women. In addition, osteoporosis also may result from nutritional and endocrine imbalance, hereditary disorders and a number of malignant transformations.

It is an object of the present invention to develop methods and compositions for generating bone in a patient who has suffered a bone fracture in an individual who, for example, is afflicted with a disease which decreases skeletal bone mass, particularly where the disease causes an imbalance in bone remodeling. Another object is to enhance bone growth to repair fracture in children suffering from bone disorders, including metabolic bone diseases. Still another object is to repair fractured bone in individuals at risk for loss of bone mass, including postmenopausal women, aged individuals, and patients undergoing dialysis. Yet another object is to provide methods and compositions for repairing defects in the microstructure of structurally compromised bone, including repairing bone fractures. Thus, the invention is aimed at stimulating bone formation and increasing bone mass, optionally over prolonged periods of time, and particularly to decrease the occurrence of new fractures resulting from structural deterioration of the skeleton.

Namkung-Matthai et al., Bone, 28:80-86 (2001) discloses a rat osteoporotic model in which bone repair during the early period after fracture is studied. Early period is denoted as within 3 to 6 weeks after fracture. Kubo et al., Steroid Biochemistry & Molecular Biology, 68:197-202 (1999) also discloses a rat osteoporotic model in which bone repair during the late period after fracture is studied. Late period is denoted as about 12 weeks after fracture. These references are incorporated by reference herein in their entirety for their disclosure of osteoporosis rat model and data regarding osteoporotic bone fracture.

In another aspect, the invention is directed to methods for strengthening bone graft in a vertebrate, e.g., a mammal, by administering the genetically modified cell according to the present invention at or near the site of fracture or breakage.

Fracture healing assays are known in the art, including fracture technique, histological analysis, and biomechanical analysis, which are described in, for example, U.S. Pat. No. 6,521,750, which is incorporated by reference in its entirety for its disclosure of experimental protocols for causing as well as measuring the extent of fractures, and the repair process, particularly in osteoporotic subjects.

In therapeutic applications, should a therapeutically effective composition be administered in combination with the connective tissue cell, the TGF-β protein may be formulated for localized administration. Techniques and formulations generally may be found in Remington's Pharmaceutical Sciences, Mack Publishing Co., Easton, Pa., latest edition. The active ingredient that is the TGF-β protein is generally combined with a carrier such as a diluent of excipient which may include fillers, extenders, binding, wetting agents, disintegrants, surface-active agents, erodable polymers or lubricants, depending on the nature of the mode of administration and dosage forms. Typical dosage forms include, powders, liquid preparations including suspensions, emulsions and solutions, granules, and capsules.

Examples of other suitable pharmaceutical carriers are a variety of cationic lipids, including, but not limited to N-(1-2,3-dioleyloxy)propyl)-n,n,n-trimethylammonium chloride (DOTMA) and dioleoylphophotidyl ethanolamine (DOPE). Liposomes are also suitable carriers for the TGF protein molecules of the invention. Another suitable carrier is a slow-release gel or polymer comprising the TGF protein molecules.

The TGF-β protein may be mixed with an amount of a physiologically acceptable carrier or diluent, such as a saline solution or other suitable liquid. The TGF-β protein molecule may also be combined with other carrier means to protect the TGF-β protein and biologically active forms thereof from degradation until they reach their targets and/or facilitate movement of the TGF-β protein or biologically active form thereof across tissue barriers.

Gene Therapy for Spine Fusion

The present invention is directed to a method of fusing targeted vertebrae on a spine by administering the inventive composition to the spine area in which the vertebrae are desired to be fused. Osteogenic effective amounts of the transformed or transfected connective tissue cells, such fibroblasts, chondrocytes or osteoprogenitor cells are contacted with the defect region or an osteogenically effective area thereof, in single injection or multiple injections as optimized by the practitioner, which results in the fusion of the targeted vertebrae.

The spine is a column of bones (vertebra) stacked on top of each other, with cushioning discs (intervertebral discs) between them. In the center of this vertebral column is the spinal cord. Spinal nerves arise from the spinal cord and exit the spine through spaces between the vertebral bodies. A bulging disc or herniated disc can press on the existing spinal nerve. An unstable spinal column allows bones to slip and rub against each other, causing back pain and possible nerve damage. Changes to the bones and discs in the vertebral column from injury or degenerative disorders can cause back pain and sometimes nerve damage.

Spine fusion surgery is generally carried out on persons with gross instability of the spine (abnormal motion), severe degenerative disc disease with hypermobility, spondylolisthesis (slippage of one vertebra over another), facet (joint) disease that has not responded to other treatments, and fractures or tumors. The best candidates for spinal fusion treatment are those in which the disc is so abnormal that the space between the vertebrae has collapsed 50% or more, or has collapsed such that the surrounding bone becomes irritated.

Bone grafting, and often implants, are used to increase stability during spine fusion surgery. After portions of the intervertebral discs are removed, the vertebral bone is roughened up and shaped to accept the graft and implant. Over time the graft fuses the adjacent levels of vertebral bone to each other. When the bone fuses, the vertebrae no longer move separately. This makes the spinal column more stable. Typically, screws, plates, cages, metal rods and other implants in spine fusion surgery are also used to increase stability.

Therapeutic Composition

In another embodiment of this invention, a compound for parenteral administration to a patient in a prophylactically or therapeutically effective amount is provided that contains a TGF-β superfamily gene harboring connective tissue cell and a suitable pharmaceutical carrier.

In therapeutic applications, the connective tissue cell harboring a gene encoding a member of the TGF-β superfamily may be formulated for localized administration. In the invention, the connective tissue cell may be generally combined with a carrier such as a diluent of excipient which may include fillers, extenders, binding, wetting agents, disintegrants, surface-active agents, erodable polymers or lubricants, depending on the nature of the mode of administration and dosage forms.

The pharmaceutical forms suitable for injectable use include sterile aqueous solutions (where water soluble) or dispersions and sterile powders for the extemporaneous preparation of sterile injectable solutions or dispersion. In all cases the form must be sterile and must be fluid to the extent that easy syringability exists. It must be stable under the conditions of manufacture and storage and must be preserved against the contaminating action of microorganisms such as bacteria and fungi. The carrier may be a solvent or dispersion medium containing, for example, water, ethanol, polyol (for example, glycerol, propylene glycol and liquid polyethylene glycol, and the like), suitable mixtures thereof, or vegetable oils. The proper fluidity can be maintained, for example, by the use of a coating such as lecithin, by the maintenance of the required particle size in the case of dispersion and by the use of superfactants. The prevention of the action of microorganisms can be brought about by various antibacterial and antifungal agents, for example, chlorobutanol, phenol, sorbic acid, theomersal and the like. In many cases, it will be preferable to include isotonic agents, for example, sugars or sodium chloride. Prolonged absorption of the injectable compositions can be brought about by the use in the composition of agents delaying absorption, for example, aluminium monostearate and gelatin.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the dosage unit forms of the invention are dictated by and directly dependent on (a) the unique characteristics of the active material and the particular therapeutic effect to be achieved, and (b) the limitations inherent in the art of compounding such an active material for the treatment of disease in living subjects having a diseased condition in which bodily health is impaired.

The principal active ingredient is prepared for convenient and effective administration in effective amounts with a suitable pharmaceutically acceptable carrier in dosage unit form. In the case of compositions containing supplementary active ingredients, the dosages are determined by reference to the usual dose and manner of administration of the said ingredients.

Delivery Systems

Various delivery systems are known and can be used to administer the composition of the invention, e.g., encapsulation in liposomes, microparticles, microcapsules, recombinant cells capable of expressing the compound, receptor-mediated endocytosis, construction of a nucleic acid as part of a retroviral or other vector, etc., and may be administered together with other biologically active agents. Administration can be systemic or local.

In a specific embodiment, it may be desirable to administer the pharmaceutical compounds or compositions of the invention locally to the area in need of treatment; this may be achieved by, for example, and not by way of limitation, local infusion during surgery, by means of a suppository, or by means of an implant, said implant being of a porous, non-porous, or gelatinous material, including membranes, such as sialastic membranes, or fibers.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. The following examples are offered by way of illustration of the present invention, and not by way of limitation.

### EXAMPLES

### Example 1

### Experimental Procedure for Bone Regeneration

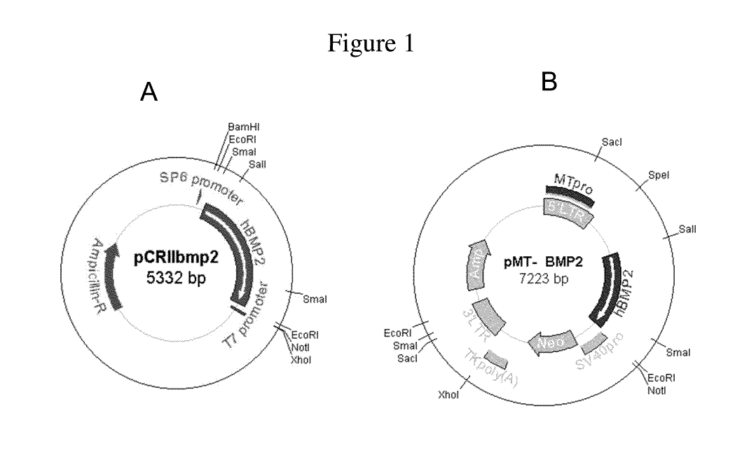

Human BMP2 gene was cloned by PCR (polymerase chain reaction) with human fetal brain cDNA and two primers. 5′ primer was 5′-TCCCAGCGTGAAAAGAGAGACTGC-3′ (SEQ ID NO:1) and 3′ primer was 5′-TTTTGCTGTACTAGCGACACCCACAACC-3′ (SEQ ID NO:2). After the PCR with GC-rich PCR system (Roche), cloning into pCRII-TOPO vector was done using TOPO TA cloning kit (Invitrogen) (FIG. 1A). For cloning into retroviral vector, pCRIIbmp2 DNA was cut with Sal I and Not I. Human BMP2 cDNA insert (˜1.2 kb) was ligated into pMTMLV with Sal I and Not I overhangs (FIG. 1B).

Packaging cell line GP-293 cell (5×10⁵ cells/p60 culture dish) was cultured one day before transfection. pMTMLV or pMT-BMP2 was transfected to GP-293 cell using Fugene (Roche). 48 hours after the transfection, neomycin was added to the culture media for the selection of neomycin resistant cells. Selection was continued for 10 days. Selected 293MT and 293MTBMP2 cells were cultured (5×10⁵ cells/p60 culture dish) for the next day's transfection of envelope coding plasmid pVSVG. 24 hours after the transfection, target cell NIH3T3 was plated for infection (1×10⁵ cells/p60 culture dish). Supernatants of transfected cells were filtered through low-protein binding filters (0.45 μm) and diluted with same volume of DMEM 48 hours after the transfection. Culture media of NIH3T3 was removed and replaced with the filtered supernatants. Polybrene was added to the final concentration of 8 μg/ml. Two days after the infection, neomycin selection was started to obtain the stable cell line of NIH3T3-neo and NIH3T3-BMP-2 cells. Selection was continued for 10 days. The amount of BMP2 produced was determined to be about 150 ng/10⁵ cells at the end of a 24 hr period.

### Example 2

### Injection of NIH3T3-BMP-2 Cells into Rabbit

New Zealand white rabbits weighing 2.0-2.5 kg were selected for animal study. The tibia bone was exposed and a defect (2 cm long and 0.5 cm deep) was made with orthopedic surgical instruments. Either control NIH3T3-neo, or NIH3T3-BMP-2 cells (2 ml of 2×10⁶ cells/ml) were injected into the defect area after suturing. At 8 weeks after injection of the cells, radiographic analysis and histological examination were performed.

### Example 3

### Weekly Radiographic Examination

New Zealand white rabbits weighing 2.0-2.5 kg were selected for animal study. The tibia bone was exposed and a defect (2 cm long and 0.5 cm deep) was made with orthopedic surgical instruments. NIH3T3-BMP-2 cells (2 ml of 2×106 cells/ml) were injected into the defect area in the tibia bone after suturing. Then radiographic analysis was performed at 1, 2, 3, 4, 5, 6, and 7 weeks after injection of the cells. The specimen was harvested at 7 weeks post injection and a picture was taken. Histological examination was carried out after harvest.

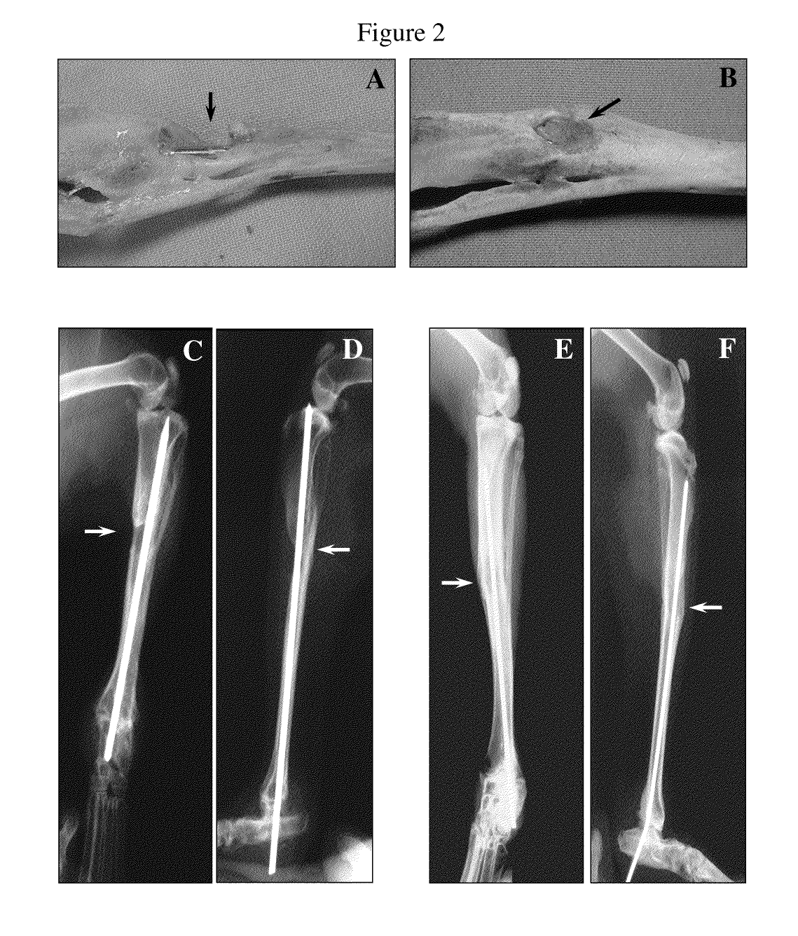

FIGS. 2A-2F show regeneration of bone with NIH3T3-BMP-2 fibroblast cells. FIGS. 1A and 1B show pictures of leg bones after 8 weeks of injection of control NIH3T3 fibroblast cells (A) and NIH3T3-BMP-2 cells (B). FIGS. 2C-2F show radiographic examinations of the control (C & D) and experimental (E & F) leg bones before sacrificing the animals. The bone defect treated with cells expressing BMP-2 proteins was healed after 8 weeks of injection whereas bone regeneration did not occur in the defect treated with control fibroblast cells.

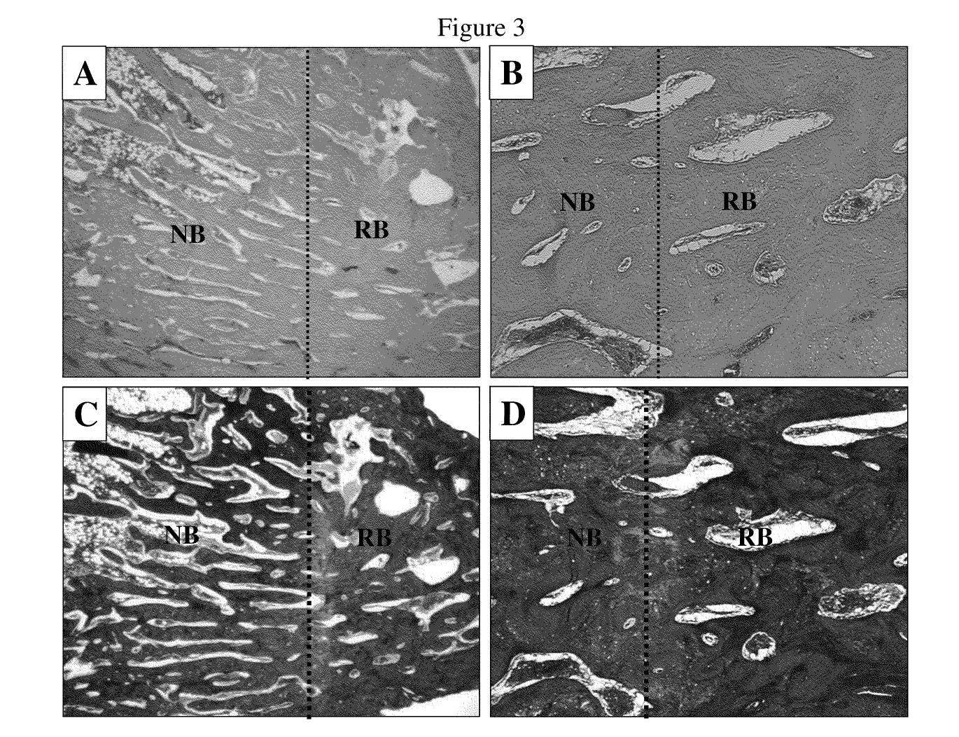

FIGS. 3A-3D show histological examination of regenerated bone tissue. Paraffin sections of the regenerated bone tissue were made and stained with Mason's trichrome. The results showed that the structure of regenerated bone tissue (RB) was almost identical to that of the normal bone tissue (NB). FIGS. 3A and 3B show low magnifications (40×), and FIGS. 3C and 3D show high magnifications (100×). The dotted line indicates the borderline between the regenerated and normal bone tissue. These results indicate that the quality of the regenerated bone is similar to that of the normal bone.

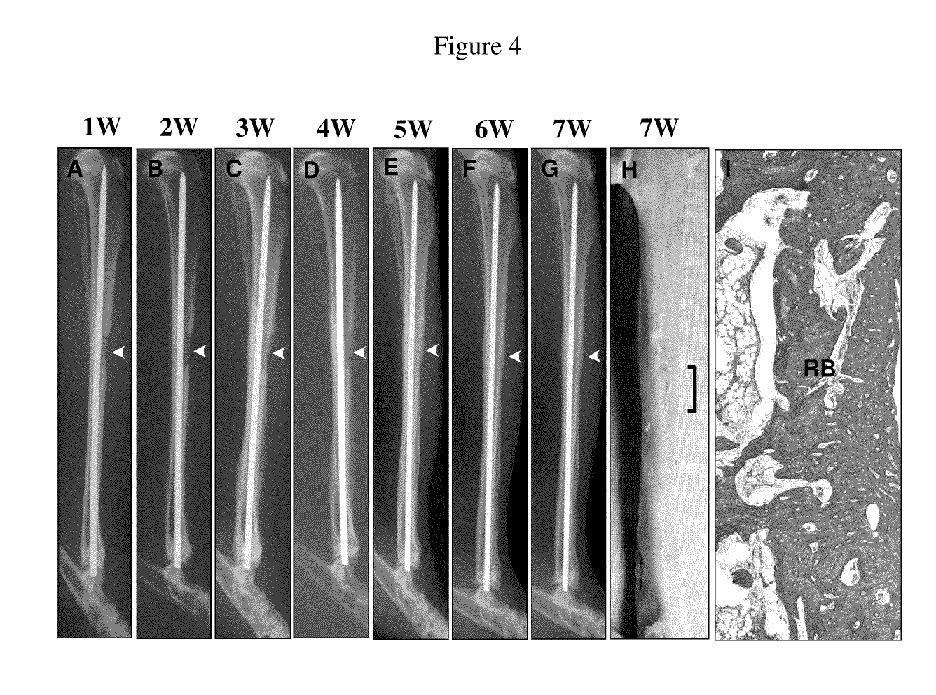

FIGS. 4A-4I show regeneration of bone with NIH3T3-hBMP2 fibroblast cells. NIH3T3-BMP-2 cells (2 ml of 2×10⁶ cells/ml) were injected into the defect area in the tibia bone after suturing. (A to G) Radiographic analysis was performed at 1, 2, 3, 4, 5, 6, and 7 weeks after injection of the cells. The results show that the defect was begun to be filled with newly generated bone tissue at three weeks after injection of the cells and completed at six weeks post injection. (H) The specimen was harvested at 7 weeks post injection and a picture was taken. This picture also shows the complete filling of the defect with regenerated bone tissue. (I) Histological examination was carried out after harvest. The results of Mason's trichrome staining is shown. Staining results indicate that the repaired bone tissue has similar characteristics as normal bone tissue.

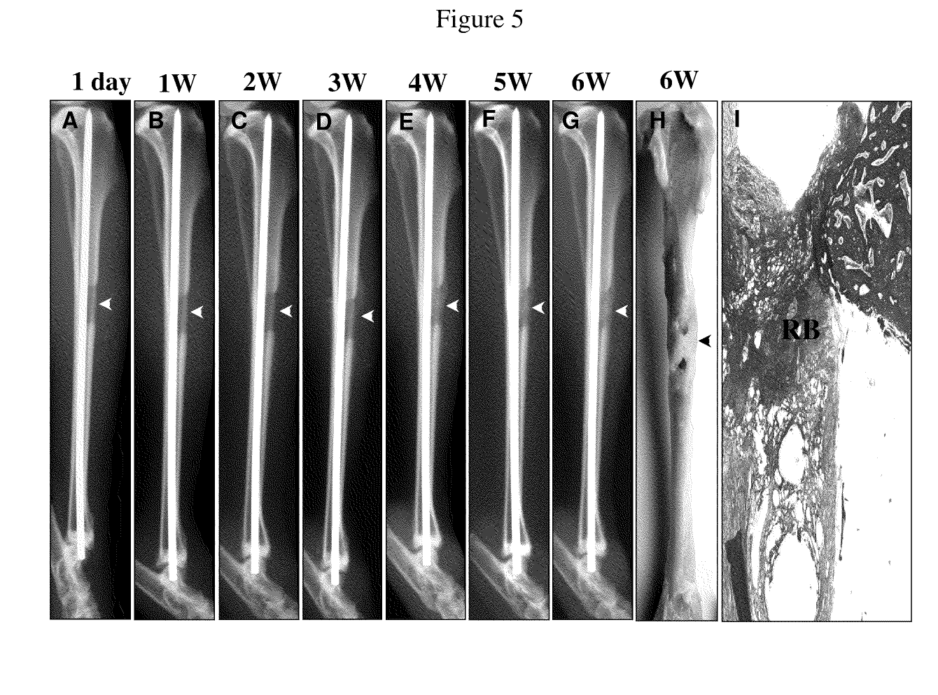

FIGS. 5A-5I show regeneration of bone with control DMEM medium. Control DMEM culture medium (2 ml) was injected into the defect area in the tibia bone after suturing. (A to G) Radiographic analysis was performed at 1 day, 1, 2, 3, 4, 5, and 6 weeks after injection of the medium. The results, in contrast to the data with NIH3T3-hBMP2 fibroblast cells, show that the defect was not filled completely even at six weeks after injection of the cells. (H) The specimen was harvested at 6 weeks post injection and a picture was taken. This picture also shows the incomplete filling of the defect. (I) Histological examination was performed after harvest. The results of Mason's trichrome staining is shown.

### Example 4

### Injection of Osteoporotic Rat with NIH3T3-BMP2

The osteoporotic model rat such as disclosed in Kubo et al., Steroid Biochemistry & Molecular Biology, 68:197-202, 1999; and Namkung-Matthai et al., Bone, 28:80-86, 2001 is used. The tibia bone is exposed and a defect (2 cm long and 0.5 cm deep) is made with orthopedic surgical instruments. Either control NIH3T3-neo, or NIH3T3-BMP-2 cells (2 ml of 2×10⁶ cells/ml) is injected into the defect area after suturing. At several weeks interval, especially at about 8 weeks after injection of the cells, radiographic analysis and histological examination are performed.

### Example 5

### Experimental Procedure for Spine Fusion

Human BMP2 was cloned and transfected into NIH3T3 as described in Example 1 above. Adherent fibroblasts from human (foreskin fibroblast derived cell line, Phase I), mouse (NIH-3T3, Phase I), rat (Lewis rat pseudarthrosis fibrous tissue derived fibroblasts, Phase II), and human (pseudarthrosis fibrous/scar tissue derived fibroblasts, Phase II) were separately cultured and transfected with BMP-2 cDNA via a retrovirus. The cells were grown using Dulbecco's Modified Eagle's Medium (Cellgro, Herndon, Va.), 10% heat-inactivated fetal bovine serum (Gibco BRL, Grand Island, N.Y.) and penicillin and streptomycin (CellGro, Herndon, Va.) in 60 mm dishes. Fibroblasts were infected for 4 hours/day for two days with a retrovirus-BMP-2 or -lacZ (negative control). ELISA was completed to determine concentration (ng/ml) of expressed protein. For each species, quantities 5×10⁶, 10×10⁶, 20×10⁶ BMP-2 producing cells were absorbed onto 1×0.5 cm collagen hemostatic sponge (ACS, Helistat, Integra LifeSciences, Plainsboro, N.J.). 0.16-0.18 mg/ml rhBMP-2 (Genetics Institute, Cambridge, Mass.) was absorbed onto 1×0.5 cm ACS (positive control). Morselized iliac crest bone was placed in the fusion site.

### Example 6

### Injection of Cells into the Spine of Rats

Total 48 female adult (3-4 months) athymic rnu/rnu rats were utilized (24 for phase I; 24 for phase II). Rats were anesthetized. A posterior midline approach was used to expose the transverse processes of L4 and L5. A high-speed burr was used to decorticate the transverse processes only. Site was irrigated (antibiotic-ringers solution). Cells/ACS were implanted between the L4 and L5 transverse processes bed. Incisions were closed. Radiographs were performed biweekly until sacrifice. L4-L5 segments were palpated manually. Motion detected between transverse processes was considered a fusion failure. Non-decalcified histology was performed.

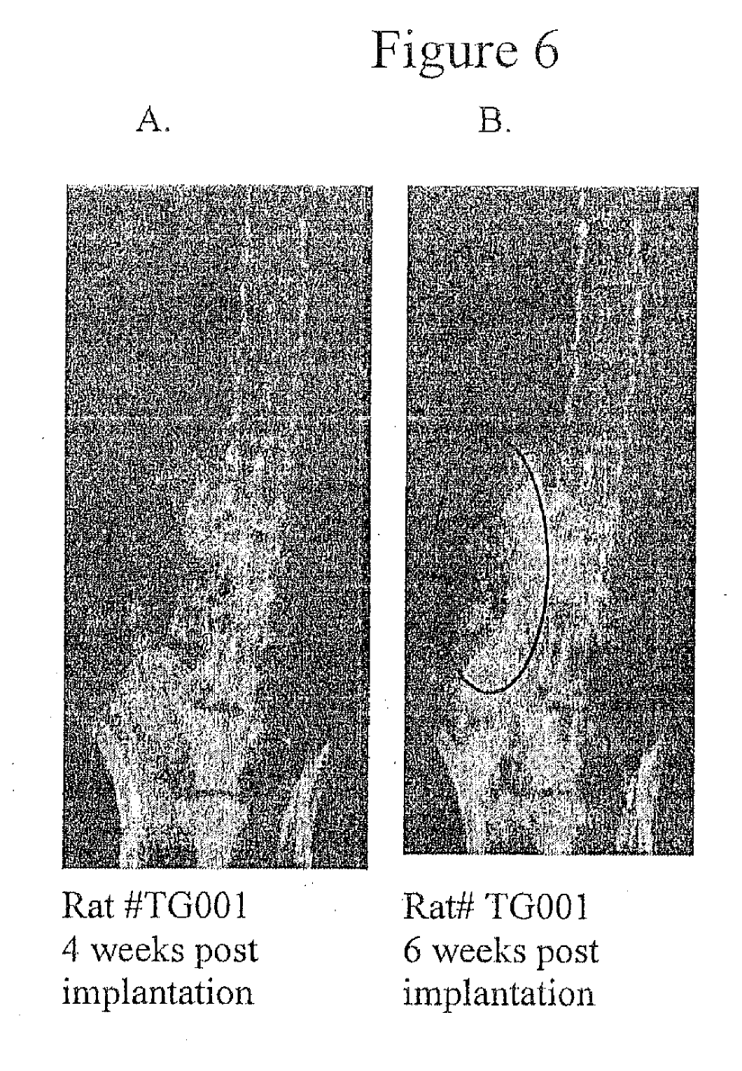

FIGS. 6A and 6B show radiographs from rat TG001, 4 and 6 weeks after posterolateral intertransverse process fusion procedure implanting cells using absorbable collagen sponge (ACS) carrier. Radiographic bridging bone on left side is encircled after 5×10⁶ fibroblasts (mouse) transfected with cDNA for BMP-2 posterolateral intertransverse process fusion study. Note less cells probably on right side and less bone formation, if any. As shown, bone is generated and fusion of the vertebrae has occurred.

### Example 7

### Materials and Methods

### Example 7.1

### Vector Construction

Each of the human genes for BMP2, BMP3, BMP4, BMP7, and BMP9 was cloned through polymerase chain reactions (PCR) using human fetal brain cDNA and gene specific primers. The genes in pCRII-TOPO (Invitrogen, Carlsbad, Calif.) were cloned into the retroviral vector pMTMLV¹⁶ (FIG. 7A).

### Example 7.2

### Construction of BMP2-Producing NIH3T3 Cells

The packaging cell line, GP2-293 cells (Clontech, Mountain View, Calif., 5×10⁵ cells/p60 culture dish), was transfected with pMTMLV or pMT-BMPs using Fugene 6 (Roche Applied Science, Indianapolis, Ind.). 293-MT and 293-MT-BMPs cells were constructed and then transfected with the envelope-coding plasmid pVSVG. NIH3T3 was used for target cells.

### Example 7.3

### Selection of BMP2 Producing Single Clones

Primary chondrocytes were isolated from the cartilage tissue that was obtained during surgical excision of a polydactyly finger from a 3-year-old female human donor. When hChonJ was used for target cells, GP2-293 cells were transfected directly with pMT-BMP2 and pVSVG and then selected. Viral supernatant from transfected GP2-293 cells was collected and filtered twice. An equal amount of Dulbecco's Modified Eagle's Medium (DMEM) with 10% fetal bovine serum (FBS) was added to the filtered supernatant in addition to 8 μg/ml of Polybrene (Sigma, St. Louis, Mo.). The viral solution was diluted 1:2 and 1:4, added to the target cells, and incubated for 4 hours. The medium was removed and replaced with normal growth medium. The infection was repeated the next day and the cells were incubated for 2 days. Infected hChonJ cells were selected with 500 μg/mL of G418 sulfate (Mediatech, Westwood, Mass.). The selection medium was changed every 2 days for 12 days. A limiting dilution method was used for single clone isolation. Confluent selected hChonJ-MTBMP2 cells were harvested and diluted. The cells were seeded at 50 cells/plate and 100 cells/plate in a 96-well plate. The cell growth was checked weekly after plating under the microscope. The medium was not changed during the single clone selection in order to check BMP2 expression of the growing colonies by collecting supernatant and performing a BMP2 assay. Only BMP2-expressing colonies were expanded to a 24-well plate and then to a six-well plate. The clones were expanded in T-75 flasks and passaged weekly.

### Example 7.4

### BMP2 Enzyme-Linked Immunosorbent Assay (ELISA)

An NIH3T3 control, 3T3-BMP2 cells, and hChonJ-MTBMP2 cells (1×10⁵) were plated in a six-well plate. The NIH3T3 control and 3T3-BMP2 cells were cultured overnight, and supernatant (20 μl/well) was collected. The hChonJ-MTBMP2 medium was changed 48 hours after seeding. Samples were collected 24 hours after the media change for the BMP2 assay. The samples then were added to an ELISA plate coated with murine monoclonal antibody against BMP2. The assay was performed according to the manufacturer's protocol (Quantikine kit, R&D Systems, Minneapolis, Minn.).

### Example 7.5

### Nude Mouse Study

This study was performed in accordance with protocols approved by the Animal Care and Use Committee of Sungkyunkwan University, Suwon, Korea. We used Balb/cAnNCrj-nu nude mice provided from the Charles River Japan Center. Nude mice weighing between 20 to 23 grams and 6 to 7 weeks of age were used in this study. The dorsal region of each mouse was injected subcutaneously with BMPs expressing NIH3T3 cell line (3T3MT-BMP2, 3T3MT-BMP3, 3T3MT-BMP4, 3T3MT-BMP7, 3T3MT-BMP9, or 3T3MT) mixed with uninfected chondrocytes (hChonJ). Nude mice also were injected with BMPs expressing NIH3T3 cell lines (3T3MT-BMP2, 3T3MT-BMP3, 3T3MT-BMP4, 3T3MT-BMP7, 3T3MT-BMP9, and/or 3T3MT) alone. Cells were injected at 1×10⁶ cells (100 μl per site. Nodule formation was observed up to 16 weeks. The mice were sacrificed when the nodule size reached about 1×1 cm. The tissues were fixed in formalin for 3 days and decalcified in nitric acid for 3 to 4 days. The blocks of specimens were embedded in paraffin and cut into 0.8-μm-thick slices. The sections were stained with hematoxylin and eosin (H&E), safranin O, and Masson's trichrome for microscopy analyses of the regenerated cartilage and bone.

### Example 7.6

### Determination of Lethal Irradiation Dose for hChonJ-BMP2A and hChonJ-BMP2E

Frozen vials of BMP2 expressing single clones hChonJ-BMP2A and hChonJ-BMP2E were thawed and the cells were washed. Cells were aliquoted into 15-ml conical tubes (2×10⁶ cells/4 ml) and irradiated with 10, 13, 15, 17, 20, or 25 Gy of gamma irradiation. The irradiated cells were spun and resuspended in 1 ml. The cells were counted and seeded into six-well plates (1×10⁵ cells/well). After the cells were incubated for 4, 8, 12, 16, 20, 24, 28, and 32 days, we determined the amount of BMP2 by ELISA. The proliferation of cells was monitored by counting cells on days 4, 8, 12, 16, 20, 24, 28, and 32. As all cells died at the radiation doses initially tested, retesting was performed to determine the proliferation of cells irradiated with 5, 10, or 15 Gy of gamma irradiation. The limit of detection for cell counting was 1×10⁴ cells/ml.

### Example 7.7

### Preparation of Osteoporotic Rats

The experimental animals used in the study were 2-month-old Sprague Dawley female rats. At about 6 to 7 weeks of age, the rats were anesthetized with lompun (3.5 mg/kg) and ketamine hydrochloride (20 mg/kg), and bilateral ovariectomies were performed from a dorsal approach. The protocol was approved by the institutional animal care and use subcommittee of the Inha University College of Medicine, Inchon, Korea. Beginning the day after ovariectomy, ovariectomized rats were given a calcium- and phosphorus-free diet for 5 weeks to provide an osteoporotic model. Water and food were available ad libitum. The calcium- and phosphorus-free diet contained casein (200 g/kg), DL-methionine (3 g/kg), corn starch (150 g/kg), corn oil (50 g/kg), sucrose (499.99 g/kg), cellulose (50 g/kg), mineral mix, AIN76 (170915) (35 g/kg), vitamin mix, AIN76A (40077) (10 g/kg), choline bitartrate (2 g/kg), and ethoxyquin (antioxidant) (0.01 g/kg). AIN76A is a supplement for the calcium- and phosphorus-free diet (supplied by Hanlive, Paju, Korea). Right tibiae of rats were scanned by high resolution in-vivo micro-computed tomography (CT) (Skyscan 1076, SKYSCAN, Belgium) at a pixel size of 18 μm. To confirm an induction of osteoporosis in rats, structural parameters and volumetric bone mineral density (vBMD, g/cm3) for trabecular bone were measured by CT-An 1.8 (SKYSCAN, Belgium).

### Example 7.8

### Injection of BMP2 Producing Cells into the Bone Fracture

To produce the fracture, rats were anesthetized with an intraperitoneal injection of ketamine hydrochloride (20 mg/kg body weight) and lompun (3.5 mg/kg body weight) and the right hind leg was shaved. A 2.5-cm incision was performed at the tuberositas tibiae and a closed midshaft fracture of the right tibia was produced using a special fracture device that produces a standardized fracture. After closed reduction, the tibiae were stabilized intramedullary with 0.9-mm titanium K wires and the incision was closed by stitching the skin layer. On day 3 after fracture, the samples were injected at the fracture site under sterile conditions. For the injection of irradiated BMP2-producing single clones, cells were taken from the liquid nitrogen storage, washed with DMEM, placed into 15-ml conical tubes (2×10⁶ cells/4 ml of DMEM), and irradiated with 10 Gy of gamma irradiation. The irradiated cells were injected into the fractures. Three osteoporotic rats were used per experimental group.

For the dose-finding study, the cells were prepared and irradiated in the same manner at dose levels of 2×10⁵, 5×10⁵, or 2×10⁶ cells/4 ml of DMEM. Five osteoporotic rats were used per group for this study.

Non-irradiated single clones (either hChonJ-BMP2A or hChonJ-BMP2E, 2×10⁶ cells) were injected at the fracture site. Untransduced human chondrocytes and medium (DMEM) were used as controls. BMP2 protein (80 μg, Anygen, GwangJu, Korea) also was used.

### Example 7.9

### Radiographic Evaluation

Posterior-anterior and lateral radiographic views were taken at 0, 2, 4, 6, 8, 10, and 12 weeks using zoom-in micro-CT, according to a method described previously.¹⁷ Computer-aided three-dimensional bone images were analyzed biweekly until week 12 as a comparison study of irradiated single clones (hChonJ-BMP2A and hChonJ-BMP2E).

### Example 7.10

### Histology in a Bone Fracture Study

After harvesting the tibia 4, 6, 8, and 12 weeks after the injection, the specimens were fixed in 10% neutral-buffered formalin (pH, 7.0) for 2 days and then decalcified in 7% nitric acid for 3 to 4 days. The blocks of specimens were embedded in paraffin wax and cut into 3-nm-thick slices. The sections were stained using Masson's trichrome for microscopy analyses of the regenerated bone.

### Example 8

### Results

### Example 8.1

### Ectopic Bone Formation of BMP2-Producing Cells in Nude Mice

The NIH3T3 cell line was modified genetically to express BMP2, BMP3, BMP4, BMP7, or BMP9 using retroviral vectors (FIG. 7) and RT-PCR showed that the relative intensities of transcription among the inserted genes were similar (data not shown). To select the most effective BMPs for bone regeneration, we examined the ectopic bone formation in nude mice injected subcutaneously with BMP-producing cells. Mice were injected with 3T3MT, 3T3MT-BMP2, 3T3MT-BMP3, 3T3MT-BMP4, 3T3MT-BMP7, or 3T3MT-BMP9 cells alone or in combination with normal untransduced chondrocytes to induce endochondral ossification. Nude mice injected with 3T3MT-BMP2, 3T3MT-BMP4, and 3T3MT-BMP9 had nodule formation regardless of the addition of normal chondrocytes (data not shown). The nodules were round and white to ivory in color. The nodules at the site of the 3T3MT-BMP2 injection were mostly white, round, and the largest in size. Formation of cartilage tissue was prominent 6 weeks after injection when human primary chondrocytes were co-injected (FIG. 7Bb). However, minimal cartilage tissue formed without the help of human primary chondrocytes (FIG. 7Ba). Cartilage tissue that formed 6 weeks after 3T3MT-BMP2 and human chondrocytes were injected converted to bone tissue at 8 weeks (FIG. 7Bd). Osteoblasts and osteoid tissues were found in the bone tissue at 8 weeks. Transformation of cartilage into bone tissue was not observed at 8 weeks when the human chondrocytes were not co-injected (FIG. 7Bc). Nude mice injected with normal-chondrocyte hChonJ did not produce nodules (data not shown). No cartilage or bone tissue formation was observed in nodules injected with 3T3-BMP4 or 3T3-BMP9.

### Example 8.2

### Selection of Single Clones hChonJ-BMP2A and hChonJ-BMP2E

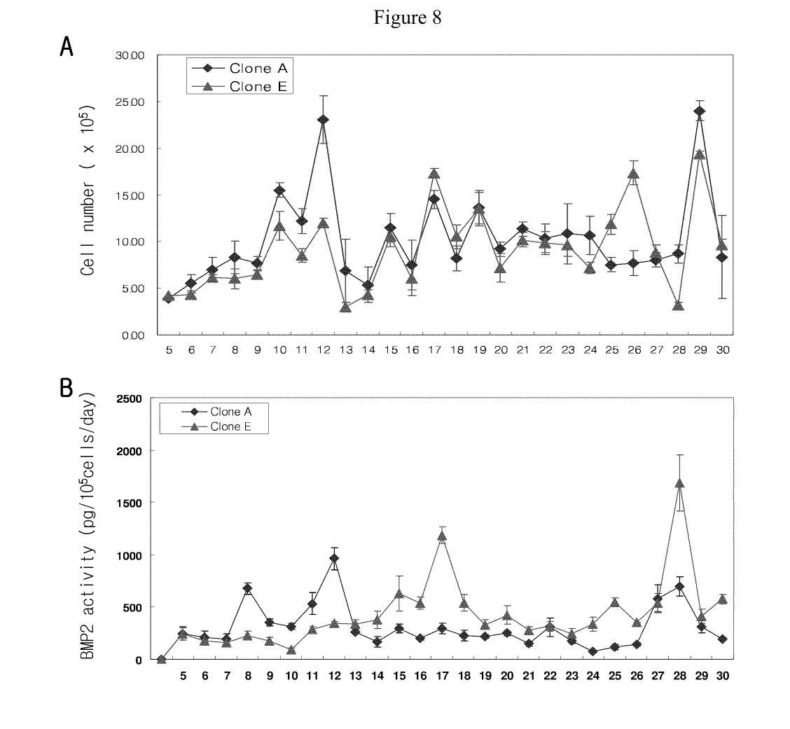

The retroviral vector pMT-BMP2 was introduced into the human primary chondrocyte hChonJ. Single clones (hChonJ-BMP2A and hChonJ-BMP2E) were selected from the infected chondrocytes using the limiting dilution method. After selection, the growth characteristics and BMP2 expression were monitored with increasing passages. Both clones showed consistent growth rates up to passage 30 (FIG. 8A). BMP2 expression from both clones was maintained at a consistent level through passage 30 (FIG. 8B).

### Example 8.3

### Determination of Lethal Irradiation Dose of hChonJ-BMP2 Single Clones

Single clones (hChonJ-BMP2A and hChonJ-BMP2E) were irradiated with one of several doses of gamma radiation (10, 13, 15, 17, 20, or 25 Gy). We determined the minimum dose of radiation required to render the replication incompetent by evaluating cell growth for 8, 12, 16, 20, 24, 28, and 32 days. With all radiation doses, both clones began dying off 2 weeks after irradiation, and all cells were dead 25 days after irradiation. The BMPs expression levels from the irradiated clones were checked every 4 days for 32 days after the irradiation (FIG. 9A). Interestingly, BMP2 production increased from the time of irradiation for 2 weeks with all radiation dose levels. The amount of BMP2 expression in hChonJ-BMP2E was 878 pg/10⁵ cells/day 4 days after irradiation. BMP2 production increased significantly until it reached the maximum level of 2,322 pg/10⁵ cells/day 12 days after irradiation. Subsequently, the BMP2 expression level decreased gradually until it was undetectable approximately 1 month after irradiation. Single clones (hChonJ-BMP2A and hChonJ-BMP2E) were irradiated with one of several doses of gamma radiation (10, 13, 15, 17, 20, or 25 Gy). Because the cells died at all radiation levels tested, retesting was performed to check the proliferation rate of the irradiated cells. Single clones (hChonJ-BMP2A and hChonJ-BMP2E) were irradiated with a lower dose of gamma radiation (5 Gy), and two doses of gamma radiation (10 and 15 Gy) (FIG. 9C). For both clones, the cell numbers increased from the date of incubation when 5 Gy was used for irradiation. When the dosage was increased to 10 or 15 Gy, the cell numbers decreased from the date of incubation in both clones. Cells irradiated with 10 or 15 Gy were not detected after 12 days of incubation.

### Example 8.4

### Healing of Osteoporotic Bone Fractures after Injection of BMP2-Producing Cells in Rats

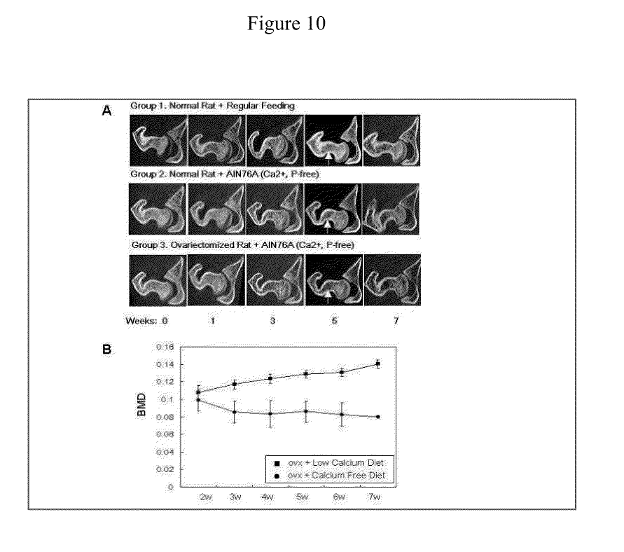

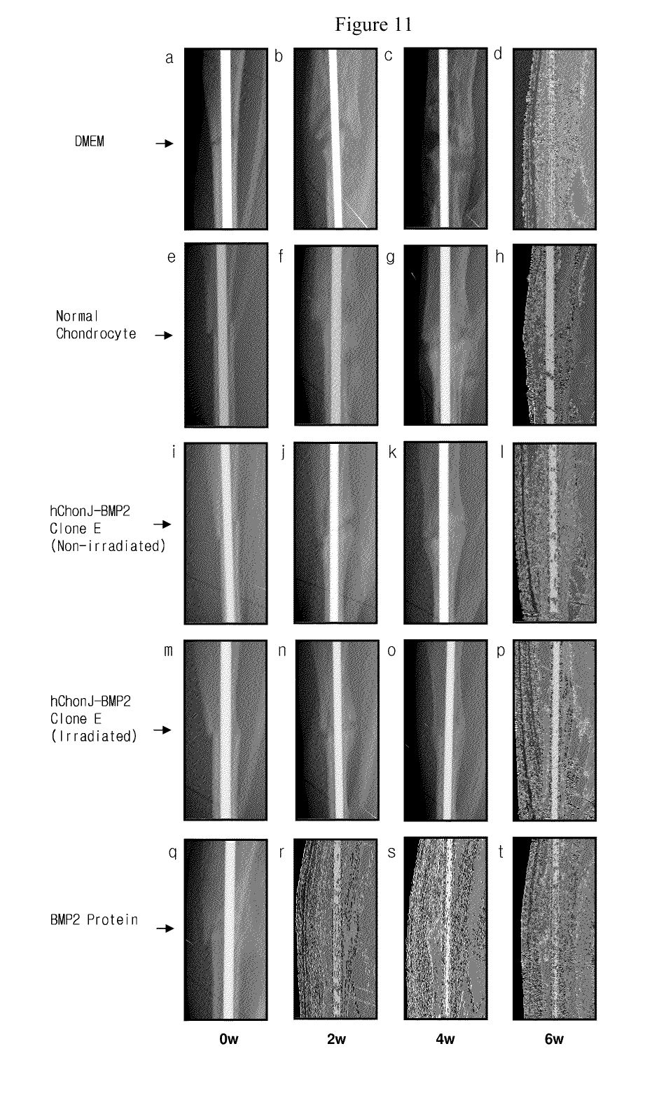

To study the effect of BMP2 produced from the irradiated cells on the repair of osteoporotic bone fracture, we generated osteoporotic rats by ovariectomy combined with a calcium- and phosphorus-free diet. The BMDs of ovariectomized rats fed a calcium- and phosphorus-free diet were lower than that of the ovariectomized rats fed a regular diet (FIG. 10B). Reduced bone density also was confirmed with micro-CT scans (FIG. 10A). Each selected single clone (hChonJ-BMP2A and hChonJ-BMP2E) was injected into a fracture area on the tibia of an osteoporotic rat. Radiographic analysis was performed 0, 2, 4, and 6 weeks after injection into the bone defects in three osteoporotic rats per group (FIG. 11). DMEM and normal chondrocytes were used for control injections. Bone fractures were identified as a horizontal gap near the fracture site on the tibia. The healing time was measured as the time to when the horizontal gap closed. The horizontal gaps closed in an average 7.7 weeks after injection when BMP2-producing non-irradiated human chondrocyte single clone (hChonJ-BMP2A or hChonJ-BMP2E) was used. In rats injected with irradiated BMP2-producing human-chondrocyte single clones (irradiated hChonJ-BMP2A or hChonJ-BMP2E), bone healing was completed in 6.3 weeks. When BMP2 protein alone was injected, bone healing finished in an average 6.5 weeks. It took an average of 9.3 and 6.3 weeks for DMEM and normal-chondrocyte controls, respectively. Compared with the tendency for thickening around the fractured tibia during the healing process as a result of overgrowth, the shape of the bones in the rats injected with irradiated hChonJ-BMP2E were almost the same as the original bones before the fractures were created (FIG. 11 m, p).

### Example 8.5

### Histology of Osteoporotic Bone Fractures Treated with BMP2-Producing Cells

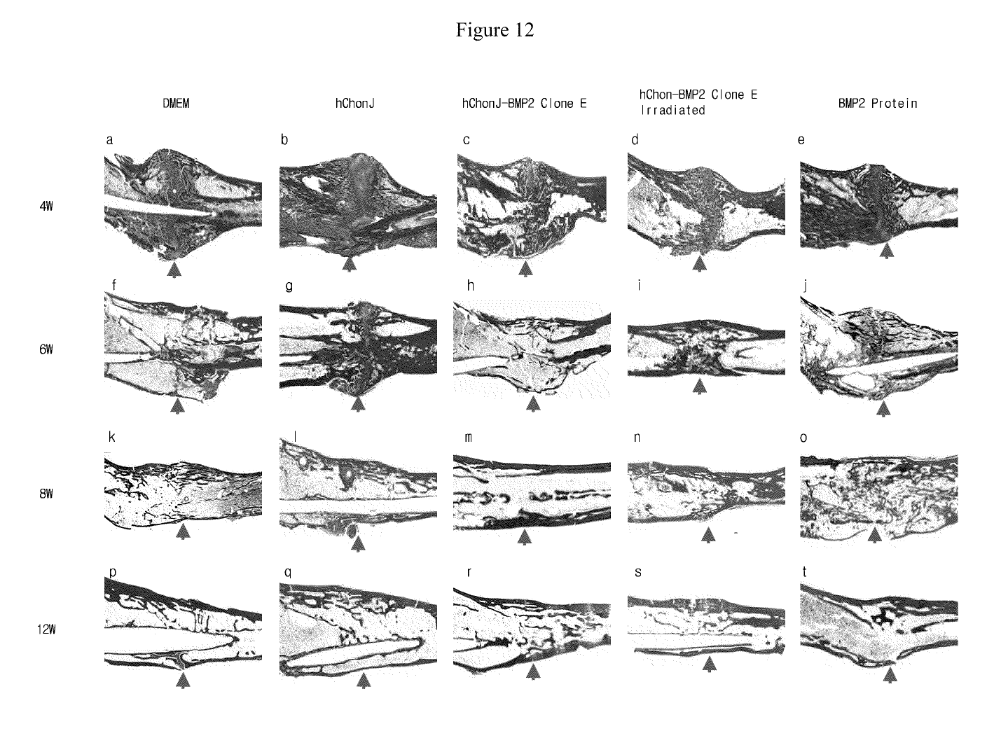

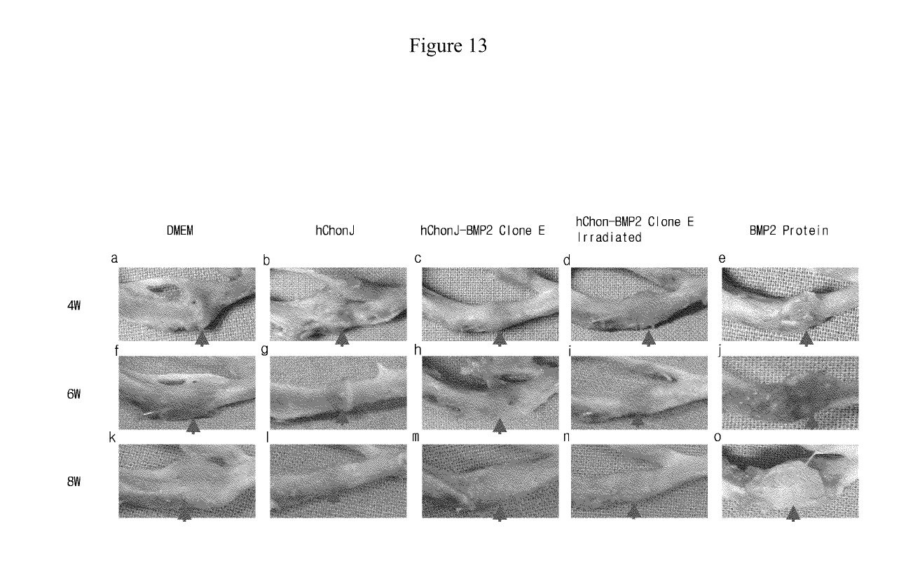

When irradiated hChonJ-BMP2E was injected (FIG. 12d), most of the gap in the tibia closed and converted into bone by 4 weeks, while areas of cartilage were still present in the filled gaps when other samples were injected (FIG. 12a, b, c, e). Six weeks after injection, bridging of the fracture gap with bone was almost finished and showed continuation of bone marrow across the fracture site (FIG. 12i). The final shapes of the bones in the rats injected with irradiated hChonJ-BMP2E were similar morphologically to the original bones before the fractures were created (FIG. 12s). The thickest compact bone, which stained blue, was in the outer space in the bone (FIG. 12s). Discontinuations in the bone at the fracture site were still present 6 weeks after human chondrocytes alone (hChonJ) was injected (FIG. 12g); with other injections, continuous bone structures were seen at the fracture sites. When BMP2 protein (80 μg) alone was injected, extra tissue formed around the callus (FIG. 13e, j, o) that were soft with numerous pores. The morphology of the regenerated bone induced by the irradiated BMP2-producing cell was most similar to original bone before the fractures were created (based on all histologic and bone images and FIGS. 12 and 13).

### Example 8.6

### Dose Comparison of Injected Irradiated BMP2-Producing Cells

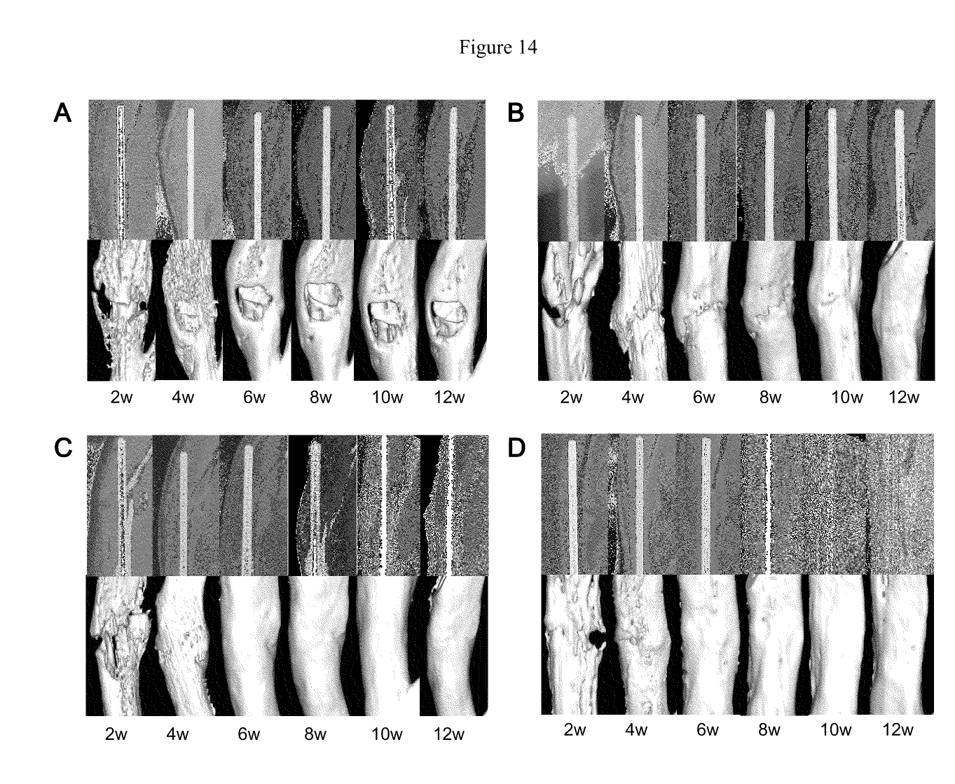

The effects of the number of cells injected into osteoporotic rats on bone regeneration were scrutinized. Different numbers of selected clone hChonJ-BMP2A and hChonJ-BMP2E (2×10⁵, 5×10⁶, and 2×10⁶) were irradiated with 10 Gy of gamma irradiation. Radiogram and computer-aided three-dimensional bone images were analyzed biweekly until week 12 (FIG. 14). The average number of weeks (7.6 weeks) for fractured bone to regenerate was shortest when the highest number of irradiated hChonJ-BMP2E was injected into the fracture.

All of the references cited herein are incorporated by reference in their entirety. Whereas particular embodiments of this invention have been described above for purposes of illustration, it will be evident to those persons skilled in the art that numerous variations of the details of the present invention may be made without departing from the invention as defined in the appended claims.

### PARTIAL LIST OF REFERENCES

1. Cornell, C. N. Internal fracture fixation in patients with osteoporosis. The Journal of the American Academy of Orthopaedic Surgeons 11, 109, 2003.
2. Cornell, C. N. Osteobiologics. Bulletin (Hospital for Joint Diseases (New York, N.Y. 62, 13, 2004.
3. Shin, M., Abukawa, H., Troulis, M. J., and Vacanti, J. P. Development of a biodegradable scaffold with interconnected pores by heat fusion and its application to bone tissue engineering. Journal of biomedical materials research 84, 702, 2008.
4. Liu, Y., Li, J. P., Hunziker, E. B., and de Groot, K. Incorporation of growth factors into medical devices via biomimetic coatings. Philosophical transactions 364, 233, 2006.
5. Larsson, S., and Bauer, T. W. Use of injectable calcium phosphate cement for fracture fixation: a review. Clinical orthopaedics and related research, 23, 2002.
6. Adkisson, H. D., Strauss-Schoenberger, J., Gillis, M., Wilkins, R., Jackson, M., and Hruska, K. A. Rapid quantitative bioassay of osteoinduction. J Orthop Res 18, 503, 2000.
7. Lecanda, F., Avioli, L. V., and Cheng, S. L. Regulation of bone matrix protein expression and induction of differentiation of human osteoblasts and human bone marrow stromal cells by bone morphogenetic protein-2. Journal of cellular biochemistry 67, 386, 1997.
8. Lee, J. Y., Musgrave, D., Pelinkovic, D., Fukushima, K., Cummins, J., Usas, A., Robbins, P., Fu, F. H., and Huard, J. Effect of bone morphogenetic protein-2-expressing muscle-derived cells on healing of critical-sized bone defects in mice. The Journal of bone and joint surgery 83-A, 1032, 2001.
9. Rutherford, R. B., Moalli, M., Franceschi, R. T., Wang, D., Gu, K., and Krebsbach, P. H. Bone morphogenetic protein-transduced human fibroblasts convert to osteoblasts and form bone in vivo. Tissue engineering 8, 441, 2002.
10. Friedlaender, G. E., Perry, C. R., Cole, J. D., Cook, S. D., Cierny, G., Muschler, G. F., Zych, G. A., Calhoun, J. H., LaForte, A. J., and Yin, S. Osteogenic protein-1 (bone morphogenetic protein-7) in the treatment of tibial nonunions. The Journal of bone and joint surgery 83-A Suppl 1, S151, 2001.
11. Swiontkowski, M. F., Aro, H. T., Donell, S., Esterhai, J. L., Goulet, J., Jones, A., Kregor, P. J., Nordsletten, L., Paiement, G., and Patel, A. Recombinant Human Bone Morphogenetic Protein-2 in Open Tibial Fractures. A Subgroup Analysis of Data Combined from Two Prospective Randomized Studies. The Journal of bone and joint surgery 88, 1258, 2006.
12. Burkus, J. K., Transfeldt, E. E., Kitchel, S. H., Watkins, R. G., and Balderston, R. A. Clinical and radiographic outcomes of anterior lumbar interbody fusion using recombinant human bone morphogenetic protein-2. Spine 27, 2396, 2002.
13. Bilic, R., Simic, P., Jelic, M., Stern-Padovan, R., Dodig, D., van Meerdervoort, H. P., Martinovic, S., Ivankovic, D., Pecina, M., and Vukicevic, S. Osteogenic protein-1 (BMP-7) accelerates healing of scaphoid non-union with proximal pole sclerosis. International orthopaedics 30, 128, 2006.
14. Tremoleda, J. L., Forsyth, N. R., Khan, N. S., Wojtacha, D., Christodoulou, I., Tye, B. J., Racey, S. N., Collishaw, S., Sottile, V., Thomson, A. J., Simpson, A. H., Noble, B. S., and McWhir, J. Bone tissue formation from human embryonic stem cells in vivo. Cloning and stem cells 10, 119, 2008.
15. Lieberman, J. R., Daluiski, A., Stevenson, S., Wu, L., McAllister, P., Lee, Y. P., Kabo, J. M., Finerman, G. A., Berk, A. J., and Witte, O. N. The effect of regional gene therapy with bone morphogenetic protein-2-producing bone-marrow cells on the repair of segmental femoral defects in rats. The Journal of bone and joint surgery 81, 905, 1999.
16. Lee, K. H., Song, S. U., Hwang, T. S., Yi, Y., Oh, I. S., Lee, J. Y., Choi, K. B., Choi, M. S., and Kim, S. J. Regeneration of hyaline cartilage by cell-mediated gene therapy using transforming growth factor beta 1-producing fibroblasts. Human gene therapy 12, 1805, 2001.
17. Chun, I. K., Cho, M. H., Park, J. H., and Lee, S. Y. In vivo trabecular thickness measurement in cancellous bones: longitudinal rat imaging studies. Physiological measurement 27, 695, 2006.
18. Vaccaro, A. R., Patel, T., Fischgrund, J., Anderson, D. G., Truumees, E., Herkowitz, H. N., Phillips, F., Hilibrand, A., Albert, T. J., Wetzel, T., and McCulloch, J. A. A pilot study evaluating the safety and efficacy of OP-1 Putty (rhBMP-7) as a replacement for iliac crest autograft in posterolateral lumbar arthrodesis for degenerative spondylolisthesis. Spine 29, 1885, 2004.
19. Luu, H. H., Song, W. X., Luo, X., Manning, D., Luo, J., Deng, Z. L., Sharff, K. A., Montag, A. G., Haydon, R. C., and He, T. C. Distinct roles of bone morphogenetic proteins in osteogenic differentiation of mesenchymal stem cells. J Orthop Res 25, 665, 2007.
20. Kochanowska, I., Chaberek, S., Wojtowicz, A., Marczynski, B., Wlodarski, K., Dytko, M., and Ostrowski, K. Expression of genes for bone morphogenetic proteins BMP-2, BMP-4 and BMP-6 in various parts of the human skeleton. BMC musculoskeletal disorders 8, 128, 2007.
21. Li, J. Z., Li, H., Sasaki, T., Holman, D., Beres, B., Dumont, R. J., Pittman, D. D., Hankins, G. R., and Helm, G. A. Osteogenic potential of five different recombinant human bone morphogenetic protein adenoviral vectors in the rat. Gene therapy 10, 1735, 2003.
22. Cheng, H., Jiang, W., Phillips, F. M., Haydon, R. C., Peng, Y., Zhou, L., Luu, H. H., An, N., Breyer, B., Vanichakarn, P., Szatkowski, J. P., Park, J. Y., and He, T. C. Osteogenic activity of the fourteen types of human bone morphogenetic proteins (BMPs). The Journal of bone and joint surgery 85-A, 1544, 2003.
23. Krebsbach, P. H., Gu, K., Franceschi, R. T., and Rutherford, R. B. Gene therapy-directed osteogenesis: BMP-7-transduced human fibroblasts form bone in vivo. Human gene therapy 11, 1201, 2000.
24. Song, S. U., Hong, Y. J., Oh, I. S., Yi, Y., Choi, K. B., Lee, J. W., Park, K. W., Han, J. U., Suh, J. K., and Lee, K. H. Regeneration of hyaline articular cartilage with irradiated transforming growth factor beta1-producing fibroblasts. Tissue engineering 10, 665, 2004.
25. Lesniak, M. S., Tyler, B. M., Pardoll, D. M., and Brem, H. Gene therapy for experimental brain tumors using a xenogenic cell line engineered to secrete hIL-2 Journal of neuro-oncology 64, 155, 2003.
26. Cohen, E. S., and Bodmer, H. C. Cytotoxic T lymphocytes recognize and lyse chondrocytes under inflammatory, but not non-inflammatory conditions Immunology 109, 8, 2003.
27. Holt, G. E., Halpern, J. L., Dovan, T. T., Hamming, D., and Schwartz, H. S. Evolution of an in vivo bioreactor. J Orthop Res 23, 916, 2005.
28. Bishop, G. B., and Einhorn, T. A. Current and future clinical applications of bone morphogenetic proteins in orthopaedic trauma surgery. International orthopaedics 31, 721, 2007.

What is claimed is:

1. A method for making bone at a bone defect site comprising:
a) inserting a gene encoding a protein having bone regenerating function into a vector operatively linked to a promoter, and
b) transfecting or transducing a population of connective tissue cells in vitro with said recombinant vector; and
c) transplanting the mammalian cell into the bone defect site, and allowing the bone defect site to make the bone.

2. The method according to claim 1, wherein said vector is a retroviral or plasmid vector.

3. The method according to claim 1, wherein said gene belongs to TGF-β superfamily.

4. The method according to claim 3, wherein said gene encodes BMP.

5. The method according to claim 4, wherein said gene encodes BMP-2.

6. The method according to claim 1, wherein said connective tissue cell is fibroblast, chondrocyte, bone progenitor cell or a combination thereof.

7. The method according to claim 1, wherein the connective tissue cells are allogeneic relative to the host mammal.

8. The method according to claim 1, wherein said connective tissue cell is irradiated before transplanting the mammalian cell into the bone defect site.

9. The method according to claim 1, wherein the bone is generated during early or late period after fracture.

10. The method according to claim 1, wherein the bone defect site is of a subject suffering from low bone mass.

11. A method of fusing a spine, comprising:
a) inserting a gene encoding a protein having bone generating function into a vector;
b) transfecting or transducing a population of connective tissue cells in vitro with said recombinant vector; and
c) contacting an osteogenic effective amount of the transfected or transduced population of connective tissue cells and a pharmaceutically acceptable carrier thereof with the spine such that expression of the DNA sequence encoding the gene at the spine results in the generation of bone, whereby the spine is fused.

12. The method according to claim 11, wherein said vector is a retroviral or plasmid vector.

13. The method according to claim 11, wherein said connective tissue cell is fibroblast, chondrocyte, bone progenitor cell or a combination thereof.

14. The method according to claim 11, wherein the connective tissue cells are allogeneic relative to the host mammal.

15. The method according to claim 11, wherein said gene belongs to TGF-β superfamily.

16. The method according to claim 15, wherein said gene encodes BMP.

17. The method according to claim 16, wherein said gene encodes BMP-2.

18. The method according to claim 11, wherein said connective tissue cell is irradiated before transplanting the mammalian cell into the spine.

19. A method of healing osteoporotic fracture comprising:
a) inserting a gene encoding a protein having bone regenerating function into a vector,
b) transfecting or transducing a population of connective tissue cells in vitro with said recombinant vector; and
c) introducing the connective tissue cell into the fracture site, and allowing the fracture to heal.

20. The method according to claim 19, wherein said vector is a retroviral or plasmid vector.

21. The method according to claim 19, wherein said gene belongs to TGF-β superfamily.

22. The method according to claim 21, wherein said gene encodes BMP.

23. The method according to claim 22, wherein said gene encodes BMP-2.

24. The method according to claim 19, wherein said connective tissue cell is irradiated before transplanting the mammalian cell into the spine.

25. The method according to claim 19, wherein the bone is generated during early or late period after fracture.

26. The method according to claim 19, wherein said connective tissue cell is fibroblast, chondrocyte, bone progenitor cell or a combination thereof.

27. The method according to claim 19, wherein the connective tissue cells are allogeneic relative to the host mammal.
